(19) United States
(12) Patent Application Publication
(10) Pub. No.: US 2026/0001015 A1
(43) Pub. Date: Jan. 1, 2026

(54) APPARATUSES AND METHODS FOR SEGREGATING SINGLE-STRANDED DNA ANALYTE MATERIAL FROM A BIOLOGICAL SAMPLE FOR ANALYSIS WITH A SENSOR DEVICE

(71) Applicant: DNA PROFILE LTD., Tel-Aviv (IL)

(72) Inventor: Roberto Igal CHERTKOW, Ashdod (IL)

(21) Appl. No.: 19/132,814

(22) PCT Filed: Nov. 14, 2023
(86) PCT No.: PCT/IL2023/051178

§ 371 Date: May 25, 2025

Related U.S. Application Data
(60) Provisional application No. 63/428,087, filed on Nov. 27, 2022.
(60) Provisional application No. 63/435,258, filed on Dec. 25, 2022.

(51) Int. Cl. B01D 39/16; C12N 15/10

(52) U.S. Cl. CPC B01D 39/1692; C12N 15/10; B01D 2239/0435; B01D 2239/0654

(57) ABSTRACT

An apparatus for transferring charged molecules between a first solution compartment and a second solution compartment includes: a barrier arranged between the first and second solution compartments: a valve configured within the barrier: and a plurality of electrets within at least one of the first and second solution compartments. the plurality of electrets sized and shaped to accumulate the charged molecules at a focal zone on a first solution compartment side of the valve. The valve may include a cavity within the valve for receipt of the charged molecules, wherein rotation of the cavity relative to the barrier causes delivery of the charged molecules within the cavity from the first solution compartment side of the valve to a second solution compartment side of the valve. The valve may be made of a conductive material and include a conductive extension opposite the cavity.

### RELATED APPLICATIONS

This Application claims priority to U.S. Provisional Patent Application No. 63/428,087, filed Nov. 27, 2022, entitled “Passive Sensor Capable of Detection of Materials Including Specific DNA or DNA-Like Strands,” and to U.S. Provisional Patent Application No. 63/435,258, filed Dec. 25, 2022, entitled “Passive Sensor Capable of Detection of Materials Including Specific DNA or DNA-Like Strands,” the contents of which are incorporated by reference as if fully set forth herein.

### FIELD OF THE INVENTION

The present invention relates to the sensing of biological and DNA-like molecules. In particular, the present invention relates to a multi-chambered implementation of such a sensor device. More particularly, the present invention relates to apparatuses and methods for transferring material from a biological sample between adjacent compartments, each compartment having a different solution for acting on the biological sample and for isolating single-stranded DNA from the biological sample, in order to enable testing of the isolated DNA strands.

### BACKGROUND OF THE INVENTION

Various methods are available for the detection of RNA, DNA, or DNA-like molecules. Such methods include gel electrophoresis and binding the DNA to DNA-specific fluorescent dyes. These methods generally require transporting the sample to a laboratory with dedicated equipment and highly qualified personnel. These methods are too inefficient and too expensive to be useful on a mass scale.

As personalized treatments are developed for people with specific DNA sequences or microbiomes, it is becoming increasingly desirable to detect specific DNA or DNA-like sequences. For example, capabilities are being developed to enable matching between the genomic properties of an individual and the microbiome of the individual, and food or cosmetic products that are best adapted for the individual.

There is accordingly a need for a simple, low-cost, disposable, passive sensor, which does not require an internal power source, for detecting cDNA/DNA/RNA like molecules.

In addition, there is a need for an implementation of this sensor that is low-cost, easy to manufacture, and relies on passive principles for isolating the cDNA/DNA/RNA-like molecules from the biological sample from which they were obtained.

### SUMMARY OF THE INVENTION

The present disclosure teaches a sensor capable of conducting tests of a material in general and RNA/DNA/DNA-like molecules in particular, without the involvement of highly trained, expensive personnel using costly laboratory equipment. The sensor is simple, cheap, and disposable. In addition, the sensor does not require an on-sensor power supply, complex electronic microchips, a microprocessor, multiplexers, and complex receivers-transmitters. The sensor may be implemented, inter alia, in the fields of microbiome and personalized beauty, to enable matching between the genomic properties of an individual or his microbiome and food or cosmetic products that are best adapted to that individual.

In particular, the present disclosure teaches a sensor that is comprised of a plurality of adjacent compartments. Each compartment contains different solutions that may serve to break down the biological material, ultimately into single-stranded DNA samples. Valves are configured between the compartments. The valves operate to transfer the analyte DNA strands to the next compartment while leaving unwanted biological material in the previous compartment.

According to a first aspect, an apparatus for transferring charged molecules between a first solution compartment and a second solution compartment is disclosed. The apparatus includes: a barrier arranged between the first and second solution compartments; a valve configured within the barrier; and a plurality of electrets within at least one of the first and second solution compartments, the plurality of electrets sized and shaped to accumulate the charged molecules at a focal zone on a first solution compartment side of the valve.

In another implementation according to the first aspect, the apparatus further includes a cavity within the valve for receipt of the charged molecules. Rotation of the cavity relative to the barrier causes delivery of the charged molecules within the cavity from the first solution compartment side of the valve to a second solution compartment side of the valve.

Optionally, the valve is comprised of a conductive material and includes a conductive extension opposite the cavity. First and second electrets are configured on respective portions of the barrier facing the second solution compartment. When the cavity faces the first solution compartment, the conductive extension is configured parallel to the first electret, thereby inducing a charge gradient within the valve that attracts the charged molecules to the cavity. Following rotation of the valve such that the cavity faces the second solution compartment, the conductive extension is parallel to the second electret, thereby inducing a charge gradient within the valve that repels the charged molecules from the cavity.

Optionally, the apparatus further includes a first barrier section made of isolating material configured to separate between the first electret and the valve extension, and a second barrier section made of isolating material configured to separate between the second electret and the valve extension.

In another implementation according to the first aspect, the valve comprises a plug that is movable relative to the barrier, wherein the plug comprises microchannels that permit passage of fluid therethrough, wherein movement of the plug relative to the barrier toward an interior of the first solution compartment reduces a volume of the first solution compartment, thereby inducing flow of the charged molecules through the microchannels to the second solution compartment.

Optionally, the plug is comprised of an agglomerate of one or more of: microbeads, microspheres, ceramic materials, or powdered materials.

Optionally, the apparatus further includes a spring configured on the second solution compartment side of the plug, wherein an extension of the spring causes the movement of the plug toward the interior of the first solution compartment.

Optionally, the apparatus further includes a first magnet attached to the plug, and a second magnet fixed at an opposite end of the first solution compartment relative to the plug. A magnetic attraction between the first magnet and the second magnet causes movement of the plug.

Optionally, a first electrostatic charge is attached to the plug, and a second electrostatic charge is fixed at an opposite end of the first solution compartment relative to the plug. An electrostatic force between the first electrostatic charge and the second electrostatic charge causes movement of the plug.

Optionally, the plug is made of conductive material.

Optionally, the plurality of electrets comprise an electret layer configured within the first solution compartment, wherein the electret layer is configured to: (1) repel the charged molecules from the end of the first solution compartment toward the plug, (2) induce a dipole within the plug; and (3) following inducing of the dipole within the plug, exert an electrostatic force on the plug to thereby: draw the plug toward an interior of the first solution compartment, reduce the volume of the first solution compartment, and induce flow of the charged molecules through the microchannels to the second solution compartment.

Optionally, the induced dipole of the plug repels the charged molecules within the second solution compartment from the plug.

In another implementation according to the first aspect, the valve comprises a plug that comprises microchannels that permit the passage of fluid therethrough; the plug is made of conductive material. The plurality of electrets comprise an electret layer configured within the first solution compartment. The electret layer is configured to: (1) repel the charged molecules from the end of the first solution compartment toward the plug, and (2) induce a dipole within the plug. The apparatus further comprises a sample holder comprised of a plurality of beads having an electric charge, the plurality of beads being bonded together with a dissolvable adhesive. Upon introduction of the sample holder holding the sample into the first solution compartment: a solution within the first solution compartment dissolves the adhesive, to thereby release the plurality of beads into the first solution compartment; and the electret induces movement of the charged molecules to the plug, diffusion of the charged molecules through the plug, and movement of the released beads to the plug.

Optionally, the beads comprise a surface that is at least partially dissolvable and an adhesive configured within the surface. Dissolution of the surface releases the inner adhesive, allowing the beads to thereby bond together at the plug to block the passage of the charged molecules backward through the plug.

In another implementation according to the first aspect, the first solution compartment contains one or more chemicals configured to break down a biological sample into double-stranded or single-stranded DNA.

In another implementation according to the first aspect, the apparatus further includes, within the second solution compartment, a sensor configured to test for the presence of specific single-stranded strands of DNA.

According to a second aspect, a method of transferring charged molecules between a first solution compartment and a second solution compartment is disclosed. The method includes: delivering the charged molecules to a focal zone of a valve configured within a barrier between the first and second solution compartments, through the operation of a plurality of electrets within at least one of the first and second solution compartments; and transferring the charged molecules through the valve.

In another implementation according to the second aspect, the valve includes a cavity therein for receipt of the charged molecules, and the method further includes rotating the cavity relative to the barrier to thereby deliver the charged molecules within the cavity from a first solution compartment side of the valve to a second solution compartment side of the valve.

Optionally, the valve member is comprised of a conductive material and includes a conductive extension opposite the cavity. First and second electrets are configured on respective portions of the barrier facing the second solution compartment. When the cavity faces the first solution compartment, the conductive extension is configured parallel to the first electret, thereby inducing a charge gradient within the valve that attracts the charged molecules to the cavity. Following the rotating step, the valve extension is parallel to the second electret, thereby inducing a charge gradient within the valve that repels the charged molecules from the cavity.

Optionally, the valve comprises a plug that is movable relative to the barrier. The plug comprises microchannels that permit passage of fluid therethrough, and wherein the transferring step comprises moving the plug relative to the barrier toward an interior of the first solution compartment, to thereby reduce a volume of the first solution compartment, inducing flow of the charged molecules through the microchannels to the second solution compartment.

Optionally, the method further includes causing movement of the plug through the extension of a spring.

Optionally, the method further includes causing movement of the plug through a magnetic attraction between a first magnet attached to the plug and a second magnet fixed at an opposite end of the first solution compartment relative to the plug.

Optionally, the method further includes causing movement of the plug through an electrostatic force induced between a first electrostatic charge attached to the plug, and a second electrostatic charge fixed at an opposite end of the first solution compartment relative to the plug.

Optionally, the plug is made of conductive material.

Optionally, the plurality of electrets comprise an electret layer configured within the first solution compartment, and the method further comprises, through the action of the electret layer: repelling the charged molecules from the end of the first solution compartment toward the plug, inducing a dipole within the plug;

and following inducing of the dipole within the plug, exerting an electrostatic force on the plug to thereby draw the plug toward the electret layer, reduce the volume of the first solution compartment, and induce flow of the charged molecules through the microchannels to the second solution compartment.

In another implementation according to the second aspect, the valve comprises a plug that comprises microchannels that permit passage of fluid therethrough. The plug is made of conductive material; and the plurality of electrets comprise an electret layer configured within the first solution compartment. The electret layer is configured to: (1) repel the charged molecules from the end of the first solution compartment toward the plug, and (2) induce a dipole within the plug. The method further comprises: receiving a sample within the first solution compartment on a sample holder comprised of a plurality of beads having an electric charge, the plurality of beads being bonded together with a dissolvable adhesive, dissolving the adhesive with a solution within the first solution compartment, to thereby release the plurality of beads into the first solution compartment; and with the electret layer, inducing movement of the charged molecules to the plug, diffusion of the charged molecules through the plug, and movement of the released beads to the plug.

In another implementation according to the second aspect, the method further includes, within the first solution compartment, breaking down biological material into double-stranded or single-stranded DNA.

In another implementation according to the second aspect, the method further includes, within the second solution compartment, testing a sample for the presence of specific single-stranded DNA strands.

### BRIEF DESCRIPTION OF THE DRAWINGS

FIGS. 1A-1H illustrate steps of isolation of single-stranded DNA from a biological sample, according to embodiments of the present disclosure;

FIGS. 2A-2B illustrate variations on the steps depicted in FIGS. 1A-1H, according to embodiments of the present disclosure;

FIGS. 3A-P illustrate apparatuses and methods for transferring material from a biological sample between adjacent solution compartments of a “smart vial,” according to embodiments of the present disclosure,

FIGS. 4A-B illustrate the transfer of biological material between adjacent solution compartments using a rotating valve, according to embodiments of the present disclosure,

FIGS. 5A-F illustrate the transfer of biological material between adjacent solution compartments using a conductive valve, in which the electrostatic effect is induced by an electret, according to embodiments of the present disclosure,

FIGS. 6A-C illustrate the transfer of biological material using a valve configured as a porous plug having microchannels therethrough, according to embodiments of the present disclosure,

FIGS. 7A-B illustrates methods for actuating the transfer of charged material through the porous plug of FIGS. 6A-C, according to embodiments of the present disclosure;

FIGS. 8A-F illustrate the transfer of biological material through a porous plug that is conductive, according to embodiments of the present disclosure; and

FIGS. 9A-E illustrate the automatic transfer of biological material through a plug using charged beads, according to embodiments of the present disclosure.

### DETAILED DESCRIPTION OF THE INVENTION

The present invention relates to the sensing of biological and DNA-like molecules. In particular, the present invention relates to a multi-chambered implementation of such a sensor device. More particularly, the present invention relates to apparatuses and methods for transferring material from a biological sample between adjacent compartments, each compartment having a different solution for acting on the biological sample, and for isolating single-stranded DNA from the biological sample, in order to enable testing of the isolated DNA strands.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways. In particular, throughout this disclosure, when the disclosure described that an element “may” be present, it is understood that the described element is not necessarily present, and the element may be replaced with an equivalent element or not be present at all. Likewise, when a list of specific examples is given, the list is not necessarily exclusive, even without a disclaimer such as “including but not limited to,” and other suitable examples may be utilized.

The sensor device described herein, in certain embodiments, is configured for sensing of DNA or DNA-like samples. Throughout the remainder of this disclosure, the analyte will be described as “DNA,” with the understanding that the sensor device described herein may be used for sensing other materials generally, and specifically other types of DNA-like materials, such as RNA or fragments thereof. The principles described herein are also applicable for isolating any biological or non-biological material for sensing, with appropriate modifications. In advantageous embodiments, the DNA samples are collected from a bodily sample, for example, saliva or cheek swab. The vial may be used for the detection of specific DNA or DNA-like strands (e.g., RNA), whether of cells of a person, of the microbiome, or of a virus. The DNA samples are then isolated, through the action of chemical solutions, within the sensor device, and brought to a sensing element within the sensor device.

In particular embodiments, the sensors described herein are configured for sensing specific single-stranded DNA strands. The principle of operation of these sensors is described at length in the aforementioned applications, which are incorporated by reference as if fully set forth herein. In brief, the sensors each contain one or more specific synthetic strands of single-stranded complementary DNA (sscDNA). When strands of ssDNA from a sample come into contact with the sscDNA of the sensor, they hybridize to the single-stranded DNA of the sensor to create double-stranded DNA (dsDNA). The sensor is configured to measure differences in physical properties that result from the hybridization of the sscDNA. For example, the ssDNA of the sensor may be configured between plates of a capacitor that is part of a resonance circuit. Bonding of the ssDNA of the analyte to the sscDNA of the sensor causes a change in the capacitance of the capacitor and, as a result, a change in the resonance frequency of the resonance circuit. The resonance circuit may act as a band stop filter for particular frequencies sent and received by a transmitter-receiver. In alternative embodiments, the single-stranded DNA of the sensor is configured between adjacent waveguides of an optical coupler, and the bonding of the ssDNA of the analyte to the sscDNA of the sensor changes the wavelengths of light that are able to be coupled across the optical coupler. The sensor device and the system on which it dwells further includes means (e.g., detectors) that are able to monitor these physical changes.

In order to ensure that the sensor is able to effectively distinguish between solutions in which the analyte DNA is not present, versus solutions in which the analyte DNA is present but is unable to reach the DNA in the sensor, it is preferable to isolate the analyte DNA from other materials. This may be accomplished by various techniques, such as gel electrophoresis. However, many such techniques require expensive laboratory equipment and trained technicians. The present disclosure introduces a device that is low-cost, and that may be operated by a layperson, that is capable of isolating ssDNA from a biological sample. This device includes various solutions for breaking down a biological sample (e.g., saliva or cheek swab) into ssDNA, and for isolating the DNA strands from the rest of the biological material. The device specifically includes different compartments, each containing different solutions for breaking down the biological sample, and with valves in between each compartment.

The device further includes means for drawing the analyte DNA to the region of the valve so that the analyte DNA is preferentially transferred from each compartment to the next compartment, while the other material from the biological sample remains behind. In many examples illustrated herein, these means are electrets. As used in the present disclosure, an electret is a material into which a pole is introduced, such that into one of its surfaces a positive charge arises, and, if designed so, at the opposite surface a negative charge originates. In general, an electret is prepared from a sheet of polymer by applying a high voltage to the polymer film while the film is being heated, so that the molecules shift, thus creating the charge distribution. Because a DNA molecule has an inherent negative charge, a positive pole of the electret attracts the DNA molecule, while a negative pole of the electret repels the DNA molecule. Alternatively, a single-poled material with positive charges may be used to attract the negatively charged species and bring them into contact with the attached molecules in the sensing zone.

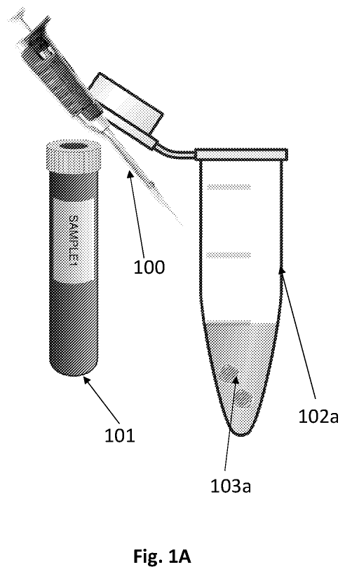

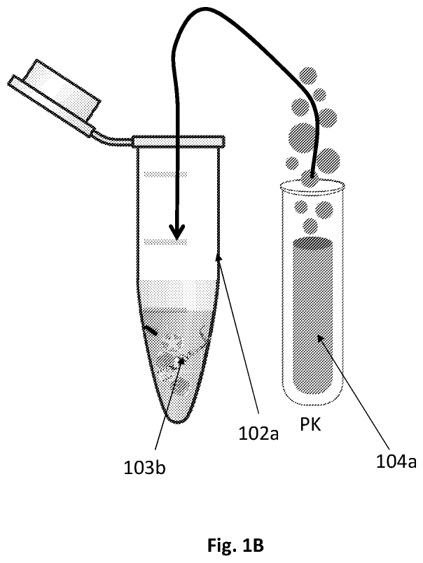

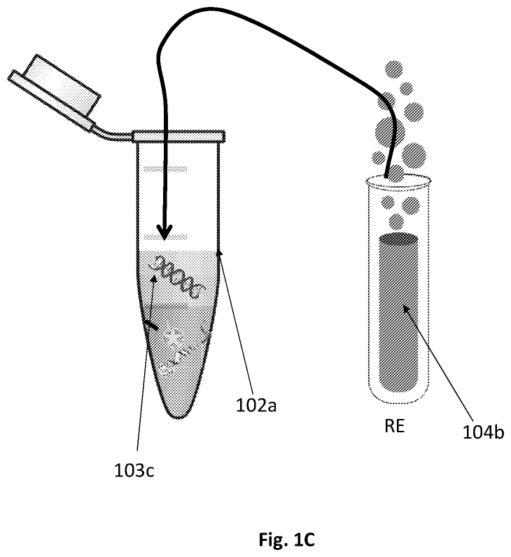

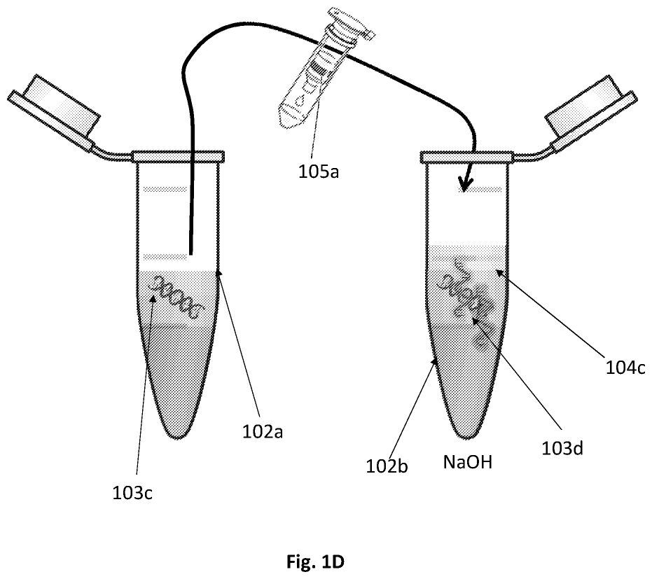

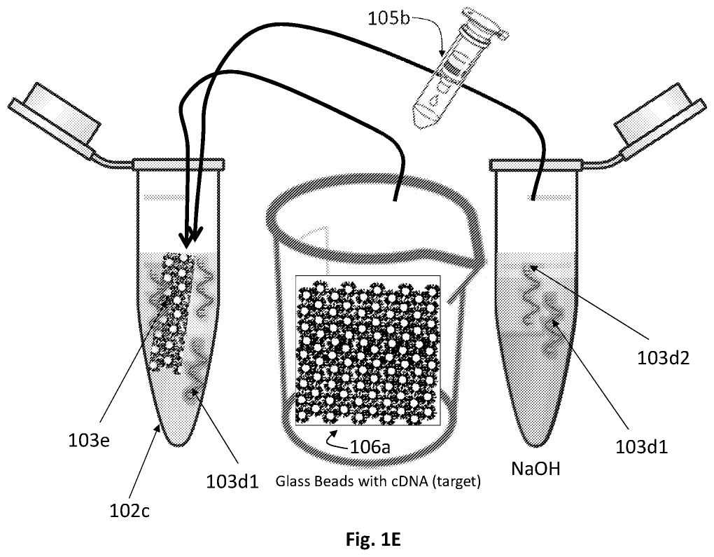

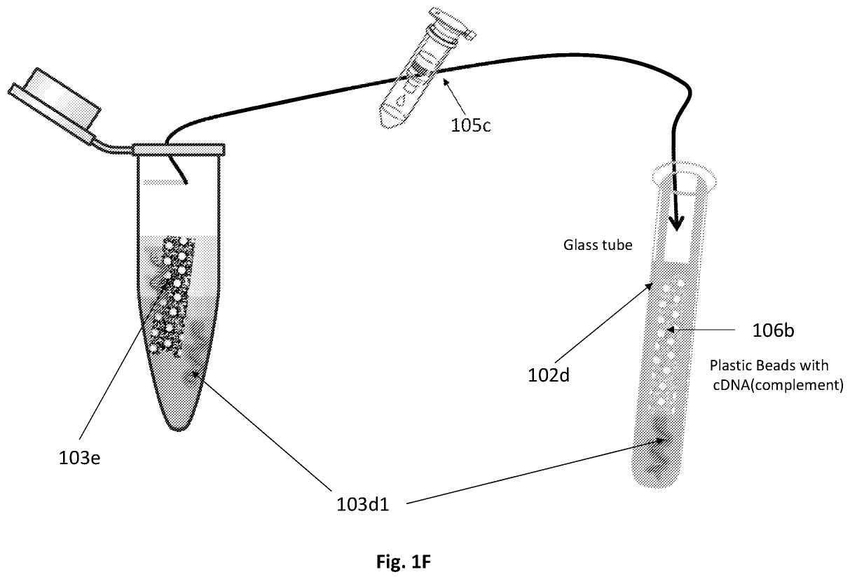

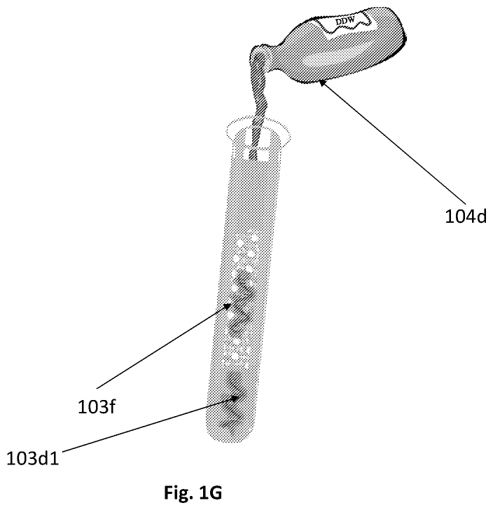

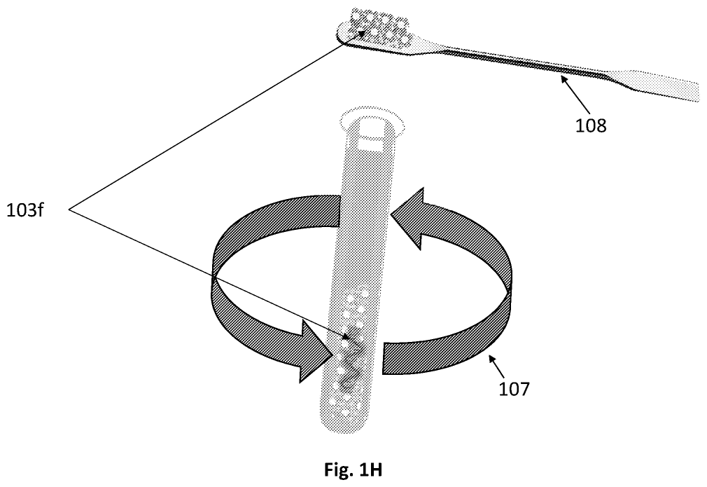

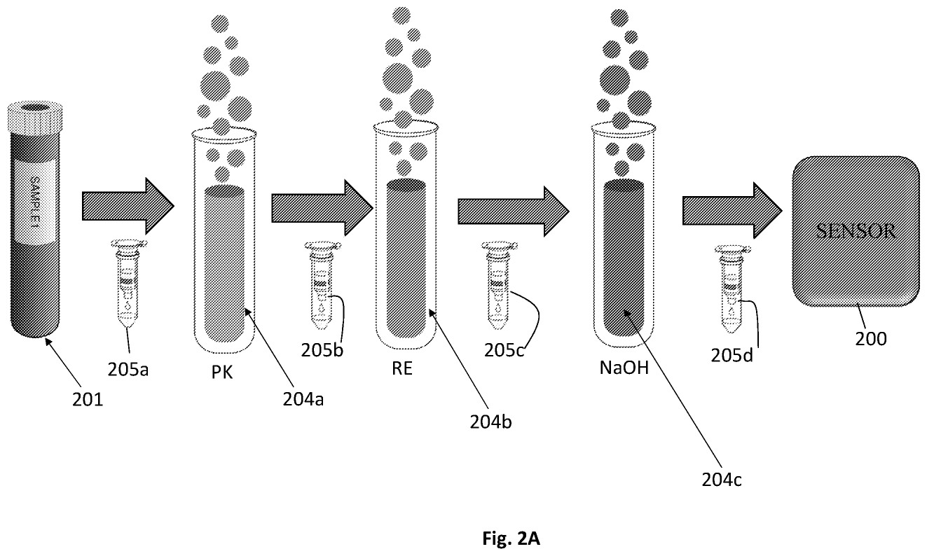

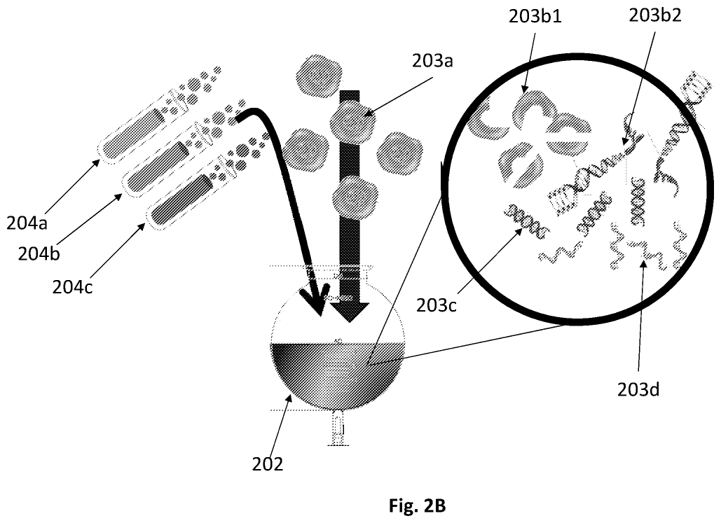

In reference to FIGS. 1A-H, the process required to prepare the sample for sensing is generally disclosed. The sequence starts by taking a sample into container 101 from an individual. The sample thus taken is transferred by some means, which in the figure is depicted by a pipette tip 100, into a vial 102a. The vial is preferably made of some polymer, or any other material. Sample 103a may contain some liquid substances and some species, such as cells or other components possibly existing in the sample, such as saliva or blood. In the next step, shown in FIG. 1B, a dissociating substance 104a, such as protein kinase (PK) is brought into contact with the sample, for example, by pouring it into the vial. The dissociating substance PK breaks the cell wall and nucleus wall (in case it exists) of the biological material and releases the DNA molecule 103b and/or RNA molecules. Once the DNA molecule is released, another substance 104b is poured to come into contact with the solution in vial 102a. This solution contains restriction enzyme (RE) 104b, which is designed to cut the DNA into fragments 103c, according to predefined criteria, as shown in FIG. 1C. At this point, optionally, per FIG. 1D, the solution in vial 102a is filtered 105a by a filtering porous media which eventually allows keeping in the solution only the required fragments 103c. The filtered solution is delivered into a different vial 102b with a dissociating solution such as NaOH 104c, which dissociates the double-stranded DNA fragment into single-stranded DNA fragments 103d. It should be understood that filter 105 here, as well as the embodiments of filters depicted in FIG. 2A, may look as in the Figure, but need not necessarily appear so. Many forms of filters are available that may look different and made of different materials and configurations, including looking as disks or papers or any other possible way.

FIG. 1E illustrates the sample after the fragments have been dissociated into the ssDNA fragment to be measured by the sensor 103d1 and its complementary ssDNA molecule 103d2. Again, optionally, the solution is filtered by porous media 105b to allow the passage of only ssDNA molecules of the relevant size while leaving other sizes or even uncut ds fragments behind. The filtered solution is transferred to a new vial 102c. At this point, an optional procedure is to bring micro beads 106a of silica or glass, for example, which are covered with artificial ssDNA strands which are copies of ssDNA that are to be analyzed such as strands 103d1. The beads may be of any suitable size, such as the micron scale size or even the nanoscale size. The beads may be of spherical form, but they may be made of oval, rectangular, pyramidal, or any other geometrical configuration. In addition, the beads may be made of various materials including glass, polymers, ceramics, metals, or magnetic material. It is possible to use even beads made of DNA material itself compacted to form an agglomerate or cluster of DNA material, which can also be considered as a bead, for example, by way of a compaction oligos. In general, the beads may be made hollow or may be formed of several layers of various materials or any other combination. When the beads are placed in the solution, if they are able, the ssDNA strands of the sample will tend to hybridize with the complementary molecules 103d2. This reaction is partially prevented by the presence of the dissociating solution 104c such as NaOH. However, close to the surface of the glass beads the density of the solution is reduced due to the chemical reaction between the silica and the NaOH, thus allowing the complementary strand 103d2 to hybridize to the strand on its surface, forming a new complex 103e. This will leave in the solution mainly, the ssDNA to be tested 353d1.

Now, as shown in FIG. 1F, the solution is optionally filtered again with appropriate filters 105c, leaving the ssDNA fragments to be tested 103d1 in the solution while blocking all the rest. At this point, the solution is introduced into a vial or tube preferably made of glass or silica 102d. In this tube, beads 106b are present. The beads 106b have ssDNA fragments 103d2 attached to their surfaces, said ssDNA fragments 103d2 being complementary ssDNA to the ssDNA molecules 103d1 to be tested. The tendency is for the strands in the beads and the strands in the solution to hybridize, creating a double stranded molecule. Since the solution might still contain separating solution 104c such as NaOH that prevents hybridization, some means are employed to increase the bonding effectiveness. These include the use of a glass tube 102d, which as mentioned previously, reacts with the solution and reduces its density. In addition, optionally, the means include adding a diluting substance 104d so as to increase the dilution of the separating substance and reduce its effect, thus allowing the ssDNA to bind into a new complex 103f as shown in FIG. 1G. At last, per FIG. 1H, the tube with the solution optionally can be centrifugated 107, such that the beads with the dsDNA 103f are concentrated at the bottom and can be transferred by some method 108 to the sensor.

It should be clear that the steps and sequence shown above indicate the steps and general substances, but the same goal can be achieved in some variation well known to those skilled in the art. For instance, vials and tubes were used in the above description; however, it is possible to use compartments and connections between the solutions. It is also possible to put the solutions in wet papers stacked each over the other, and also the filtering elements may be realized as a porous paper or disk etc., or as a smart wipe product. Exemplary implementations of such a smart wipe product are explicated in the previously referenced U.S. Provisional Patent Applications 63/428,087 and 63/435,258, as well as in the co-pending international patent application PCT/IL2023/051147, filed Nov. 8, 2023, entitled “Multilayered Planar Sensor for Detection of Materials Including Specific DNA Or DNA-like Strands,” the contents of which are hereby incorporated by reference as if fully set forth herein. It is also possible that instead of moving the solution from one container to the other, it will be the sample that is moved from one compartment to another through a pumping effect or under an external physical means such as an electret.

It is also clear that although the filters were exemplified in the previous Figures at certain locations, other placement of the filters are possible. For example, as shown in FIG. 2A, filters 205a are between the sample 201 and solution 204a and filters 205b are between the solution 204a and solution 204b and filters 205c are between the solution 204b and solution 204c and filters 205d are between the solution 204c and sensor 200. It is also obvious that more or less steps and solutions can be implemented as the case may need.

Also, it should be understood that although the different solutions were shown as to be implemented one after the other in some sequence, it does not need to be so. For example, in reference to FIG. 2B, all the solutions 204a, 204b, 204c are put together in the same flask/tube/vial/compartment at the same time that the sample materials, and within it, the cells 203a, are added. So, all the chemical reactions take place at once as soon as the required species appear in the solutions, such that the cells are broken down 203b1, releasing the DNA molecules 203b2 then being sliced into fragments 203c and ultimately separating into single stands 203d at the same location before being processed in the other stages.

It also should be clear that the sequence described in FIGS. 1A-H contains schematically all the major steps one after the other as would be taken in a general lab experiment, however, some steps might be performed in a different environment. For example, the step described in FIG. 1F can be done differently. In such an example, the beads 106b are already placed between the electrodes of the sensor, and the ssDNA sample 103d1 arrives into contact with the beads by the action of an electret. And of course, as mentioned earlier, all the steps may happen on papers soaked with the appropriate solutions and filters.

Among others, the isolation procedure may be performed using a multi-compartment device. Embodiments of suitable multi-compartment devices are described in connection with the subsequent Figures.

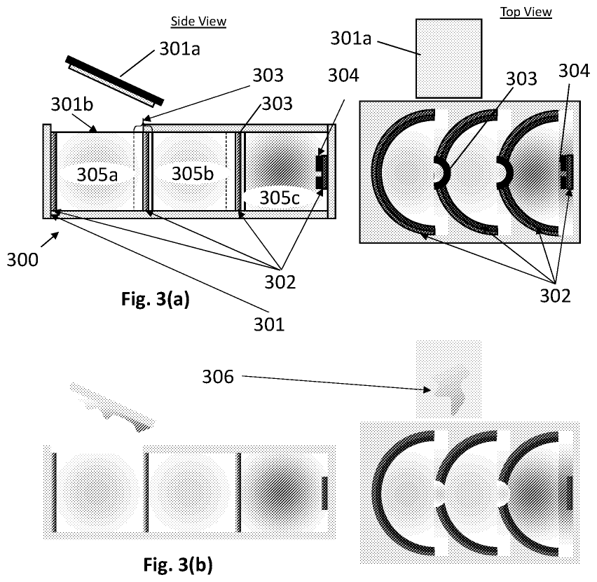

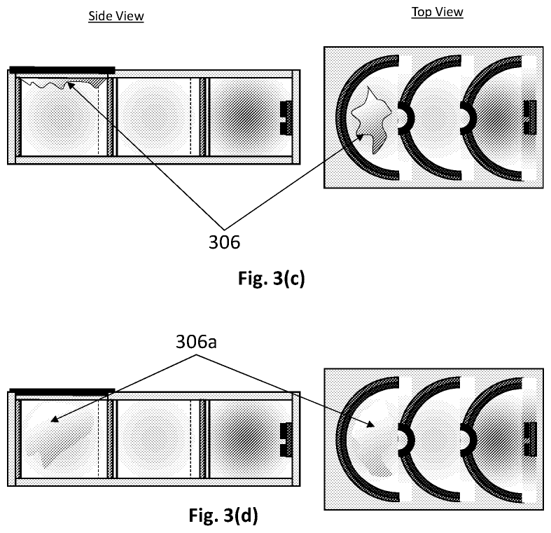

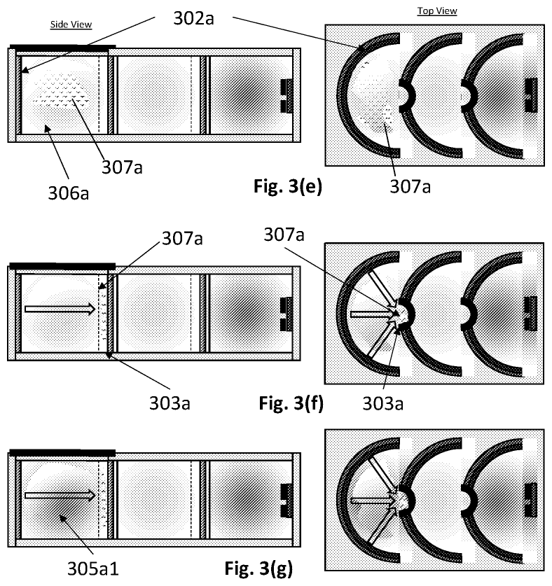

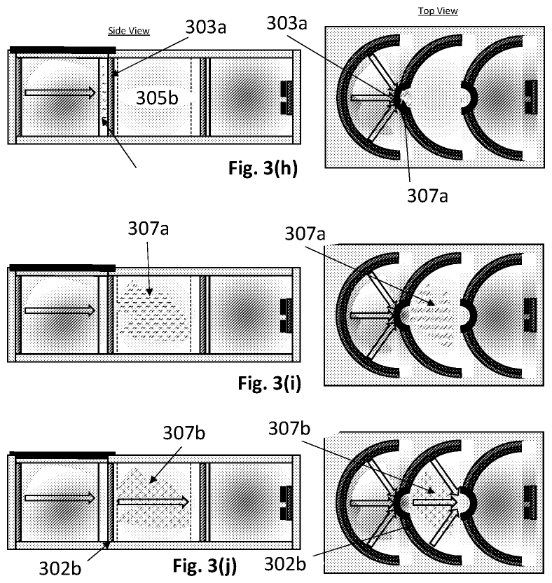

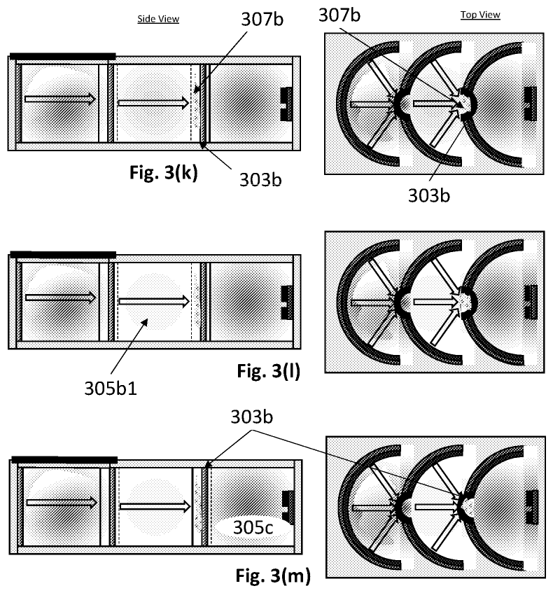

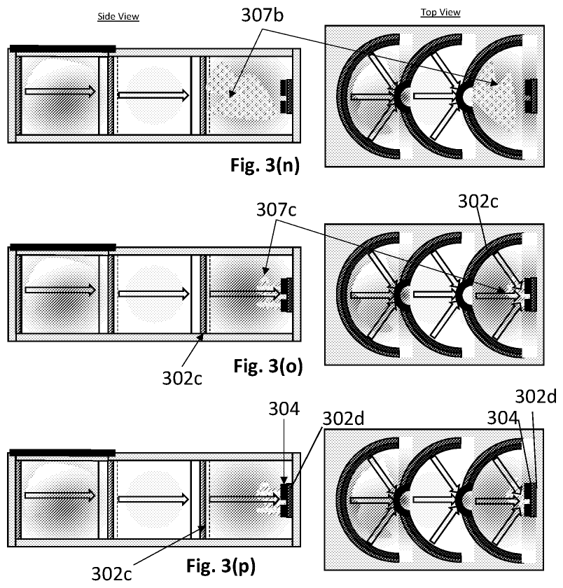

FIGS. 3A-P schematically illustrate a multi-compartment device for receiving an analyte biological sample, breaking down the biological sample into single-stranded DNA, isolating the single-stranded DNA from the rest of the biological sample, and detecting specific DNA strands. The device may be conceived as a “smart vial.” The vial includes multiple compartments, each containing chemical, electrical, and transport elements required for detection of specific DNA strands.

FIGS. 3A-P illustrate the general construction of vial 300, and of the breaking down and transfer of biological material through adjacent compartments of the vial 300. Each of FIGS. 3A-P illustrates a cross-section view (left) at the middle of the structure as seen from the top, and a top view (right) of the vial 300. Although the compartments are schematically illustrated as having the geometry of a rectangular box, the structure of each compartment may be of any suitable geometric shape, such as cylindrical, hexagonal, or asymmetric.

Vial 300 includes a plurality of compartments. In the illustrated embodiment, three compartments are shown. The three compartments hold substances 305a, 305b, 305c. These substances (also referred to herein as solutions) are configured to interact with biological samples. Although, in the examples described herein, the substances are chemical solutions, it is also possible for the substances to be gels. The gels may fill a portion of, or the entire, volume of the solution compartment. The gels may serve as a selective media for transporting certain molecules from one side of the gel to the other while blocking other molecules or blocking fluids from transferring and mixing, etc.

The respective compartments are separated by barriers. In different embodiments, the barriers may be made of conductive or nonconductive material. Valves 303 are configured within openings in the barriers and separate the contents of the respective compartments and permit the controlled transfer of substances between the compartments. Each compartment also may contain one or more electret structures 302. The electret structures may be placed at specific locations within each solution compartment, including particularly in the vicinity of the valves 303. In addition, electret structures may be placed within sensors 304.

At least one of the compartments has an opening 301b for insertion of the samples to be tested. The opening is closed by a cap 301a. The opening is presented in a schematic fashion, and other configurations for the opening are possible.

Referring to FIG. 3B, a sample 306 (for example, saliva) is placed either in cap 301a (as shown) or in corresponding hole 301b. The cap is placed so as to close the opening, as shown in FIG. 3C. This action brings the sample into contact with solution 305a of the first compartment. The sample spreads by diffusion, such as through shaking, or through other mixing means. The chemicals in solution 305a act on the sample, which will start to dissociate, to generate intermediate product 306a. In particular, DNA molecules 307a will become isolated within the solution 305a, as shown in FIG. 3E. At least initially, these DNA molecules are double-stranded. These molecules, like all DNA molecules, have negative charge sites.

Referring to FIG. 3E, electret 302a is placed at one end of the first solution compartment. Electret 302a is oriented such that its negative pole faces the DNA molecules. The negative pole of the electret repels the negatively charged DNA molecules. Furthermore, as seen in the top views, electret 302a is sized and shaped such that the electrostatic forces will repel the negatively charged molecules in a specific direction. For example, electret 302a may be shaped in a semicircular shape (as shown), a parabolic shape, or any other shape that delivers the negative charges to a focal zone. Here, the focal zone, or the place where the electret directs the negatively charged molecules, is a zone near valve 303a, as shown in FIG. 3F. The valve 303a has a cavity for receiving therein the negatively charged material.

Although, in the illustrated embodiment, the electret is configured so as to repel the charged molecules toward a focal zone, it is possible to implement other configurations in which the electrets are arranged such that the positive side is toward the molecules and they are attracted, rather than repelled, into the focal zone. Furthermore, it is possible to use a combination of electrets such that some attract some repel, as well as to install the electrets at various places in each solution compartment including the valve itself, in order to achieve the desired focusing effect.

Eventually, as shown in FIG. 3G, most of the negative-charged molecules will converge in the focal zone. Optionally, the solution in the compartment includes an indicator such that when this occurs, a color change 305a1 will be apparent, thus indicating the end of this process. At this point, valve 303a is actuated, for example through one of the processes described infra in connection with specific valve configurations. In the illustrated embodiment, valve 303a is actuated through rotation, as shown in FIG. 3H. Rotation of valve 303a relative to the barrier causes delivery of the charged molecules within the cavity from a first solution compartment side of the valve to a second solution compartment side of the valve. As a results, the contents of the valve, mainly the negative-charged DNA fragments 307a that are collected there, are blocked from solution 305a of the first compartment and exposed to solution 305b of the second compartment. Although this method of transfer is not perfectly selective, such that some materials that are not negative-charged DNA fragments may also be transferred into the second solution compartment, it is sufficiently selective to raise the concentration of DNA fragments, and to prevent the transfer of the vast majority of irrelevant material from the biological sample that is not DNA fragments into the second solution compartment. Especially when this process is repeated more than once, the resulting difference in concentration of DNA fragments significantly improves the sensitivity of the sensor. Additional specific embodiments of the valve, which may be more selective are described further herein.

In the second solution compartment, fragments 307a start to spread due to diffusion or through any other mixing process, as described above. In addition, electret 302b, which is sized and shaped in a manner similar to electret 302a, begins to act on the fragments 307a. The charged molecules eventually move toward a focal zone near valve 303b and concentrate there, as shown in FIG. 3I. In addition, as the charged molecules move within the second solution compartment, they are exposed to solution 305b. This solution contains chemicals that further act on the charged particles, converting fragments 307a into molecules 307b. Molecules 307b may be, for example, double-stranded or single-stranded DNA fragments. Because these molecules remain negatively charged, they move in the preset direction, and accumulate at the focal zone, which is near valve 303b, as shown in FIG. 3K. Optionally, it is possible to prepare indicator solution 305b such that when the molecules accumulate at the focal zone, a change in color 305b1 occurs, as shown in FIG. 3L.

Valve 303b rotates, as shown in FIG. 3M. This blocks material 307b from solution 305b in the second solution compartment, and exposes the material 307b to solution 305c of the third solution compartment. In the illustrated embodiment, the third is the final solution compartment, but, obviously, additional or fewer solution compartments may be implemented. Material 307b starts to diffuse or spread within the third solution compartment. In particular, material 307b may move due to electrostatic forces caused by electret 302c and other electrets, such as an electret within the sensor, as shown in FIG. 3N. Electret 302c has its negative pole toward the solution, which repels the negatively charged molecules to a focal zone. As previously mentioned, the electret configurations may also have a positively charged pole facing the solution, or any other configuration that will move the molecules toward the sensor. During this motion, due to the action of chemicals in solution 305c, molecules 307b transform into molecules 307c, as shown in FIG. 3O. Molecules 307c are the material that is desired to be tested, which, in the examples described herein, are single-stranded DNA. Sensor 304 is present within the third solution compartment, as shown in FIG. 3P. Sensor 304 has an electret material 302d, with its positive side facing the solution, thus attracting the negatively-charged molecules and forcing them to pass through a sensing region—for example, the gap between two plates of a capacitor. When the material 307c passes through the sensing region, strands of single-stranded DNA, if compatible with corresponding strands of single-stranded DNA configured within the sensor, will bond to the sensor single-stranded DNA. The other negatively charged molecules will pass through the sensing region and continue towards electret 302d. As previously explained, it is possible to add to solution 305c some chemical that will change to a color 305c1 when the process has ended. The sensor device will send a reading indicating the presence of analyte DNA strands, thus completing the sensing process.

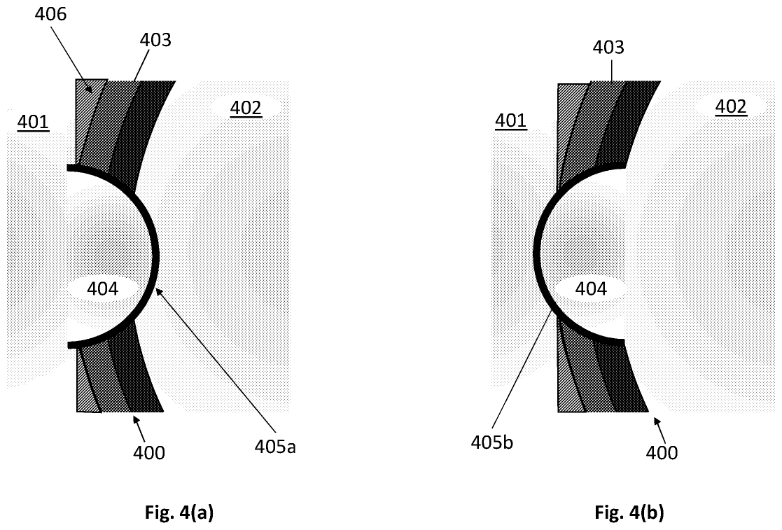

FIGS. 4A-B depict a first specific embodiment of a valve arranged between adjacent solution compartments of sensing device 400. FIG. 4A illustrates part of a first solution compartment 401 and part of a second solution compartment 402. Barrier 406 separates the solution compartments. Barrier 406 may be made of various materials, including the material of the vial itself, and may optionally be formed integral with the exterior of the vial. In this embodiment, barrier 406 is made of a non-conductive material. Electret 403 is configured adjacent to separating wall 406. The negative side of electret 403 faces second solution compartment 402. The opening between the first and second solution compartments is closed by valve 405a. Valve 405a is shown as having a hemispherical shape. As shown in FIG. 4A, valve 405a has a convex side facing second solution compartment 402 and a concave side facing first solution compartment 401. As described above, a sample that was placed in first solution compartment 401 will undergo a chemical reaction, eventually creating negatively charged molecules. Due to the action of the electret on the negatively charged species, the charged molecules will, over time, converge into a focal zone in the region of the valve. As discussed in connection with FIGS. 3A-P, this process may be implemented, at least in part, through the action of a specifically sized and shaped electret on the opposite side of the first solution compartment 401 (not shown).

Following the accumulation of the negatively charged material in the cavity of valve 405a, the cavity rotates relative to barrier 406. The concave side of the valve “scoops” or blocks solution 404 from the first solution compartment 401 and releases or exposes it at the second solution compartment 402. After this movement, the concave side of valve 405b points to the second solution compartment 402, while the convex side is now facing the first solution compartment 401, effectively blocking or closing the first solution compartment from the second solution compartment, as shown in FIG. 4B. The rotation of the cavity may proceed through any suitable mechanism. For example, it is possible to use a mechanical method such as an external knob connected to a lever that is physically moved to change the position of the valve. It is also possible to use an electrical, magnetic, or other motor connected to the valve, or a magnetic or electrostatic force imposed on the valve, or a pneumatic, hydraulic, or thermal mechanism.

Once the charged material has been transferred into the second solution compartment 402, the electret 403 may be used to direct the charged particles to the opposite end of second solution compartment 402, as discussed above. In particular, electret 403 has its positive pole facing the first solution compartment 401 (leftward in the view of FIGS. 4A-B), and its negative pole facing the second solution compartment 402 (rightward in FIGS. 4A-B). As a result, electret 403 both attracts negatively charged particles within the first solution compartment 401 to the valve region and repels negatively charged particles within the second solution compartment 402 from the valve.

It is also important to emphasize that although chemical solutions were referenced in the description, in the same places where solutions were mentioned gels may be used. The gels may be placed in the first or second compartments or even in the valve region. The gels may fill the entire volume of the compartment or valve or may extend to part of the volume. The gel may have several purposes, such as but not limited for use as a selective media to transport part of the molecules from one side to the other side of the gel while blocking other molecules or blocking the fluids from transferring and mixing between compartments or valves, and other uses that are characteristic for gels.

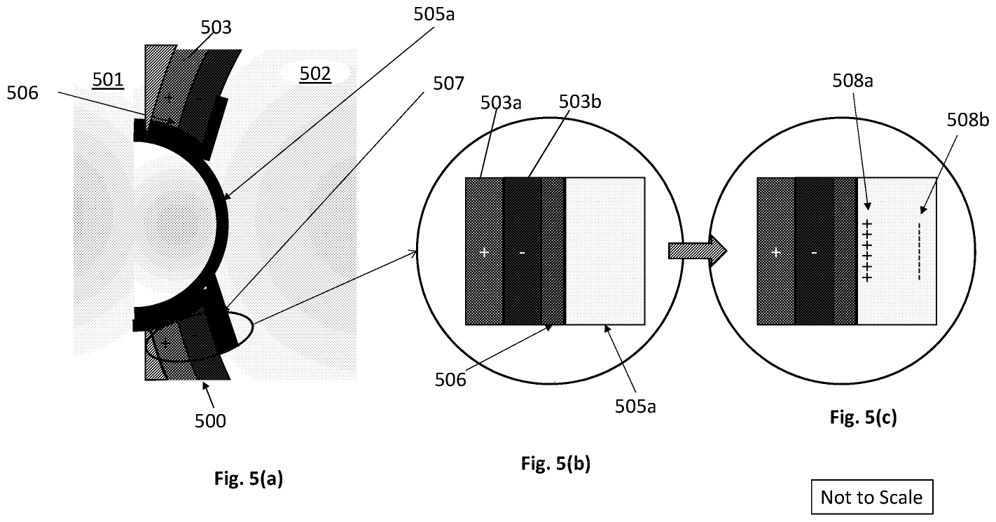

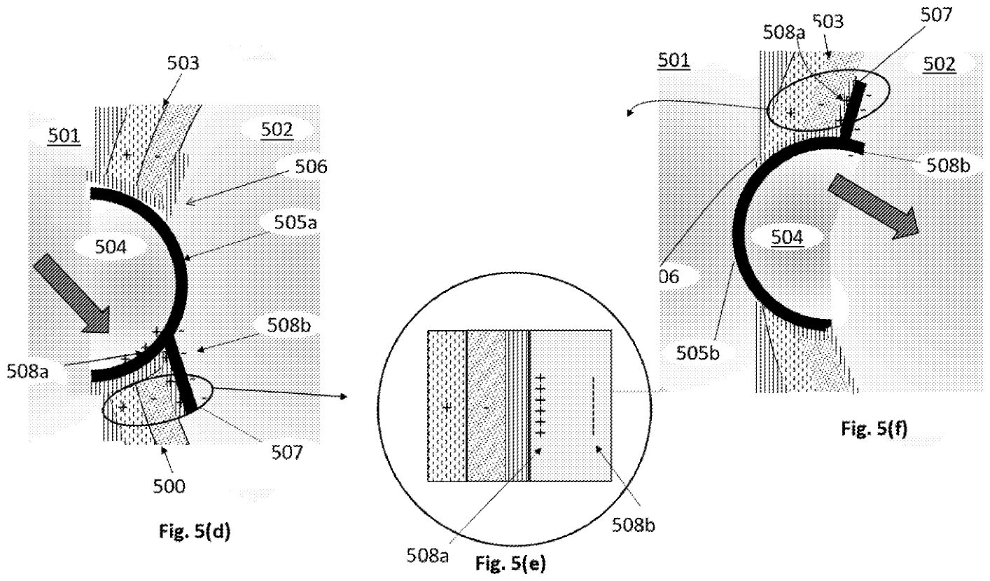

FIGS. 5A-F illustrate another embodiment of the valve. Referring to FIG. 5A, a first solution compartment 501, and a second solution compartment 502 are separated by barrier 506. Barrier 506 is preferably made of a non-conductive material but may also be conductive. Barrier 506 may be part of the structure of the vial, and may be made of the same material or a different material. An electret 503 is placed on the surface of barrier 506. Part of the electret is near an opening between solution compartments 501, 502 where valve 505a is located. The negatively charged side of the electret faces the second solution compartment 502, and the positively charged side of the electret faces the first solution compartment 502.

In the embodiment of FIGS. 5A-F, valve 505a is made of a conductive material. When barrier 506 is non-conductive, barrier 506 isolates the electret 503 from valve 505a so that the charged face of the electret is not directly connected to the conductor. However, this is not strictly necessary, and in other embodiments, it might be preferable to connect the electret to the conductive material of valve 505a.

Valve 505a is, in many respects, of a similar structure to the valve of FIGS. 4A-B and FIGS. 3A-P. One important difference between valve 505a and the previous embodiments, aside from valve 505a being made of a conductive material, is that valve 505a includes a conductive extension 507. Conductive extension 507 is used as a stopping element to prevent rotation of the valve beyond a defined angle. Thus, when the concave side is opened toward the first solution compartment 501, extension 507 is obstructed by a first section of barrier 506 and first electret 503, as shown in FIG. 5A. When the valve rotates such that the concave side faces the second solution compartment 502, as shown in FIG. 5F, extension 507 is blocked by a second section of barrier 506 and second electret 503.

In addition to serving as a blocking element that ensures the correct orientation of the valve, conductive extension 507 serves an additional purpose. Referring to FIG. 5B, a portion of barrier 506 and adjacent structures is enlarged. Within this view, it is apparent that the first and second electrets are composed of a positive side 503a and a negative side 503b, with the negative side facing the second solution compartment 502. The negative side is also partially enclosed by the first and second barrier sections 506. The barrier sections 506 are made of isolating material and are configured to separate between the first and second electrets and the conductive valve extension 507. Conductive extension 507 of valve 505a is in close contact with barrier 506. Due to electrostatic effects, the negative side 503b of the first electret induces a positive charge in the conductive extension 507 of valve 505a. This induced charge is illustrated in FIG. 5C. In addition, migration of positive charges 508a toward the negative side 503b of the electret will also cause a negative charge 508b to form on the opposite end of valve 505a. Stated differently, there is an area of valve 505a that is closer to the extension which is positively charged, and, at some distance from the extension, there is an area of valve 505a which is negatively charged.

As shown in FIG. 5D, the induced charges in the conductive valve, in the area near extension 507, will create a distribution of charges within the valve, such that mainly positive charges face the concave side of the valve, and most of the negative charges face the convex side of the valve. As a result, there is increased positive charge in the valve's concave side facing first solution compartment 501. Negatively charged material 504 in the first solution compartment is attracted to the location of increased positive charge in the valve, as indicated by the arrow. Thus, the distribution of charges in the valve supplements the activity of the electrets in focusing the negatively charged material 504 toward the valve region.

Following actuating the valve into the position of FIG. 5F, the concave side is now facing the second solution compartment 502. The valve blocks first solution compartment 501 from contacting charged material 504 or second solution compartment 502. Extension 507 has moved to a new position within the second solution compartment relative to the opening and enters into contact with second barrier section 506 and its underlying electret material 503. The second barrier section 506 ensures the correct angle or position of the valve's concave opening toward the second solution compartment 502.

In addition, due to the electrostatic effect of the negative side 503b of the second electret, positive charges 508a of the conductive valve in position 505b and of extension 507 will rearrange close to the negative electret charges. Negative charges 508b will rearrange toward the other side of the valve 505b, as shown in FIG. 5E. Although FIG. 5C and FIG. 5E appear to be identical, due to the different locations of the valve extension and the concave side of the valve relative to the electret, the end effect is different. The positive charges, which are attracted to the second electret, are, in this case, close to the convex side valve. A negative induced charge 508b is induced at the concave side of the valve, whereas, at the previous valve position 505a, a positive charge occurred. Thus, whereas in the previous valve position the induced charges of the valve attracted negative species 504, in the new configuration of valve 505b, the negative charge expels the negative species 504, as indicated by the arrow, ejecting negative molecules into the second solution compartment 502.

In sum, when the cavity faces the first solution compartment, the conductive extension 507 is configured parallel to the first electret, thereby inducing a charge gradient within the valve 505 that attracts the charged molecules to the cavity; and following rotation of the valve 505 such that the cavity faces the second solution compartment, the conductive extension is parallel to the second electret, thereby inducing a charge gradient within the valve that repels the charged molecules from the cavity.

Many variations are possible for the conductive valve 505 and extension 507 described herein. These variations include, but are not limited to, the size, the geometry, and the material of the valve; and the geometry, placement, thickness, and other technical parameters of the extension 507. These components may be arranged such that the electrostatic-induced charges fill more or less the area of the valve, either on the concave or convex sides. In addition, some zones of the electret material may be made positive and some negative, as desired for influencing the conductive material of the valve. In addition, the material of the barrier sections 506 may be thicker or thinner at various points or may have various geometries, as desired.

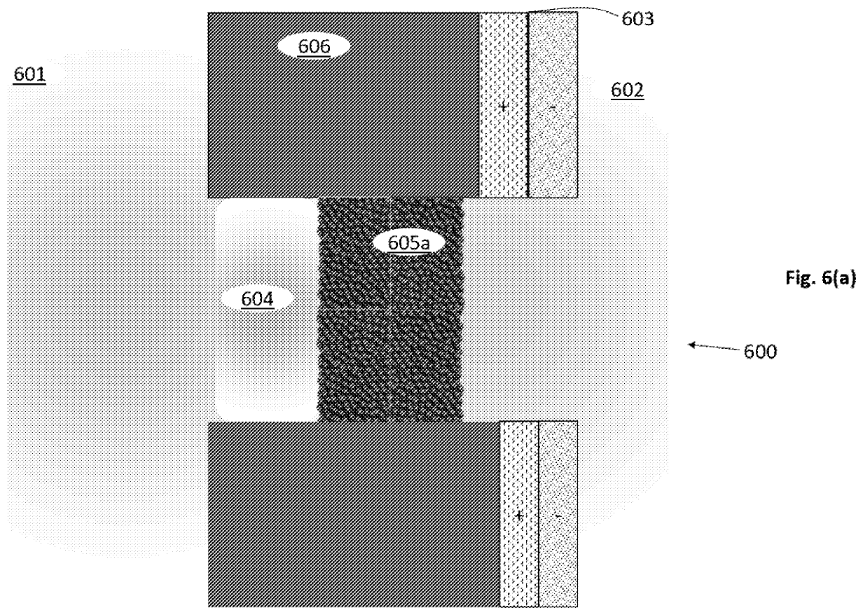

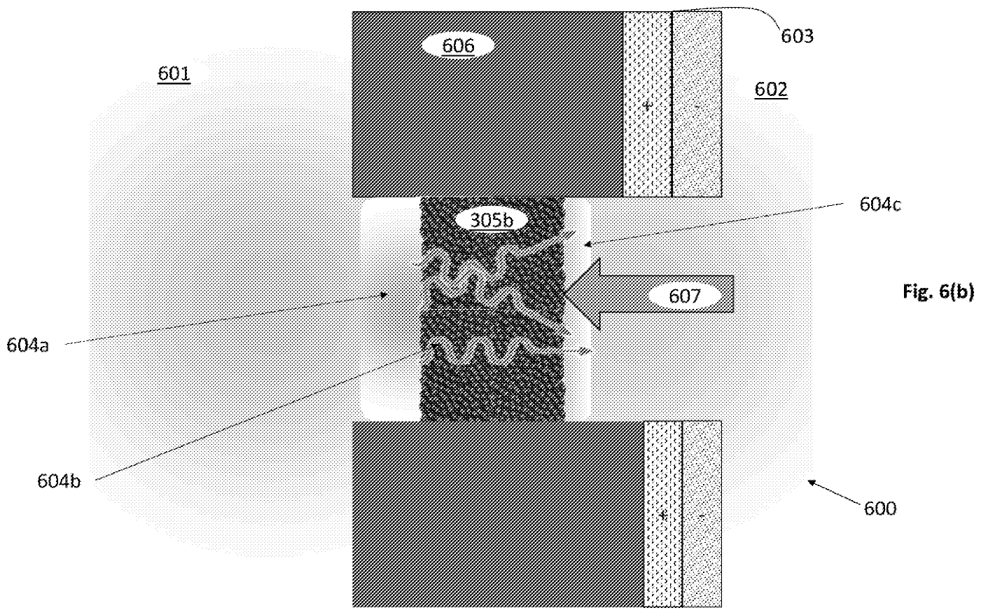

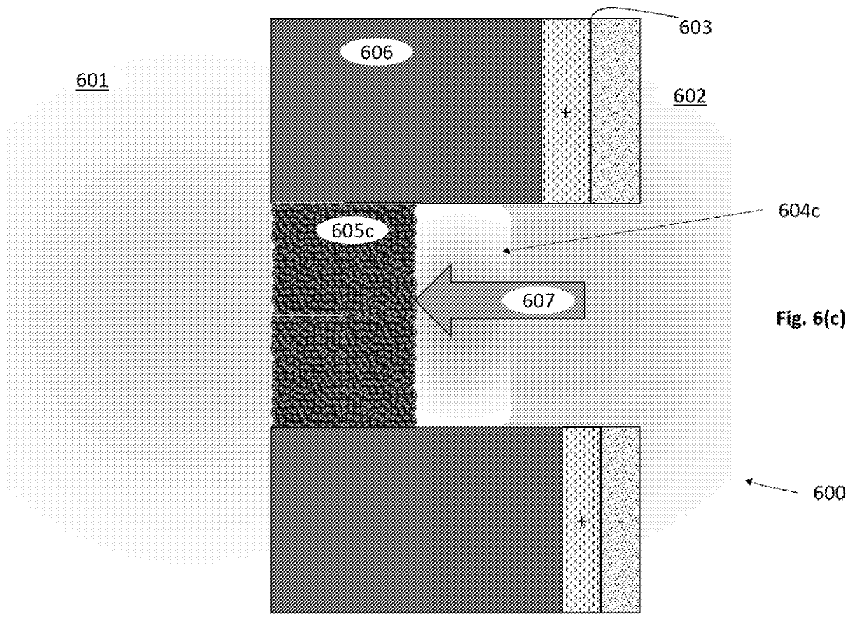

FIGS. 6A-C illustrate another embodiment of the valve. In contrast to the prior embodiments, in which the valve was fixed at a location between the first and second solution compartments, in this embodiment the valve is movable. FIG. 6A schematically illustrates a valve between compartments of device 600. A first solution compartment 601 and a second solution compartment 602 are separated by a barrier 606. In this embodiment, barrier 606 is of a nonconductive, isolating material. On the side of barrier 606 facing the second solution compartment 602, there is an electret 603. There is an opening in barrier 606 and in electret 603. Although it is illustrated in the figure as if the extent of the openings in separating element 606 and electret 603 are similar, this need not be the case, and one opening may have larger or smaller dimensions than the other.

Plug 605 is configured within the opening. In FIG. 6A, the plug is designated with reference numeral 605a. Plug 605 may be made of microbeads or microspheres packed together, as an agglomerate, a ceramic, a gel, a membrane, or any other porous material, either conductive or nonconductive, through which fluids or other substances may percolate, diffuse, flow, or transit from one side to the other. In exemplary embodiments, plug 605 is made of microbeads, which may be spherical or cubical, for example, and of the same or different sizes. The plug may also be made of powders pressed together. Plug 605 has microchannels that permit the passage of fluids therethrough, under particular conditions, for example, when suitable pressure is applied on one side of the plug. Plug 605 acts as a valve, as will be explained infra.

At the side of the plug 605a that faces first solution compartment 601, there is a region 604 at which negatively-charged material has accumulated through action of one or more electrets (not shown). This material is to be transferred to the second solution compartment, 602. As plug 605b moves, liquid 604b flows through the channels within the valve, leaving behind liquid 604a within the first solution compartment 601. Liquid 604c accumulates on the other side of plug 605. Eventually, all the material 604a is transferred through the plug 605 and is accumulated at the second solution compartment side of the plug 605. This completes the operation of the valve, effectively blocking the solution of the first compartment 601 from the solution of the second compartment 602, while allowing the controlled transfer of the material from side to side. Although the channels of plug 605 do not permit hermetic sealing, in practice, the liquids are able to change locations either by diffusion, which is a relatively slow process or through the application of a pressure difference, which is done only in a single direction.

When the negatively charged material has accumulated in zone 604, a force is applied to the plug, as illustrated in FIG. 6B. Specific ways in which this force may be applied will be described further herein. The force induces the motion of plug 605 relative to the barrier toward the interior of the first solution compartment, as indicated by arrow 607. The plug moves away from the second solution compartment 602 and into the first solution compartment 601, reducing the volume of the first solution compartment 601. Since the liquid in the compartments is not compressible (beyond some small compression due to gases possibly trapped in the solution), the advance of the plug 605 is possible only if, correspondingly, liquid flows out of first solution compartment 602 and through the channels within the plug 605. In exemplary embodiments, the fluids used to transit the plug may be superconducting fluids, such as liquid helium below the critical temperature, for example. In such instances, due to the quantum nature of the supercritical fluid, the fluid might pass through special glass materials, even without the presence of physical channels.

Although, in the illustrated embodiment, plug 605 moves relative to wall 606, which remains stationary, this motion is properly understood as a relative motion. Thus, plug 605 may be stationary relative to barrier 606 or even the entire structure of the vial, and these components may move relative to the plug, to achieve the same relative motion. Furthermore, in alternative embodiments, the plug may not move at all, and instead a reduction of pressure in the second solution compartment, or an increase in pressure in the first solution compartment, may induce the flow through the channels in the plug. In addition, although it was specified that flow occurs between a first solution compartment into a second solution compartment, this need not be so, and the solution may be made to flow in the opposite direction if so required, or even starting the flow in one direction and then proceeding with a flow in the opposite direction.

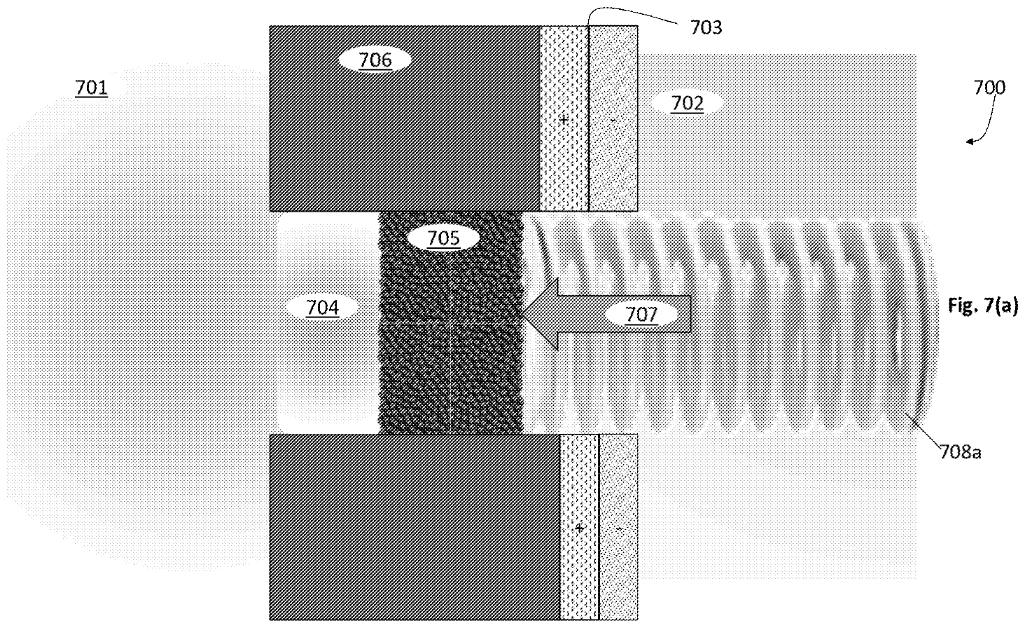

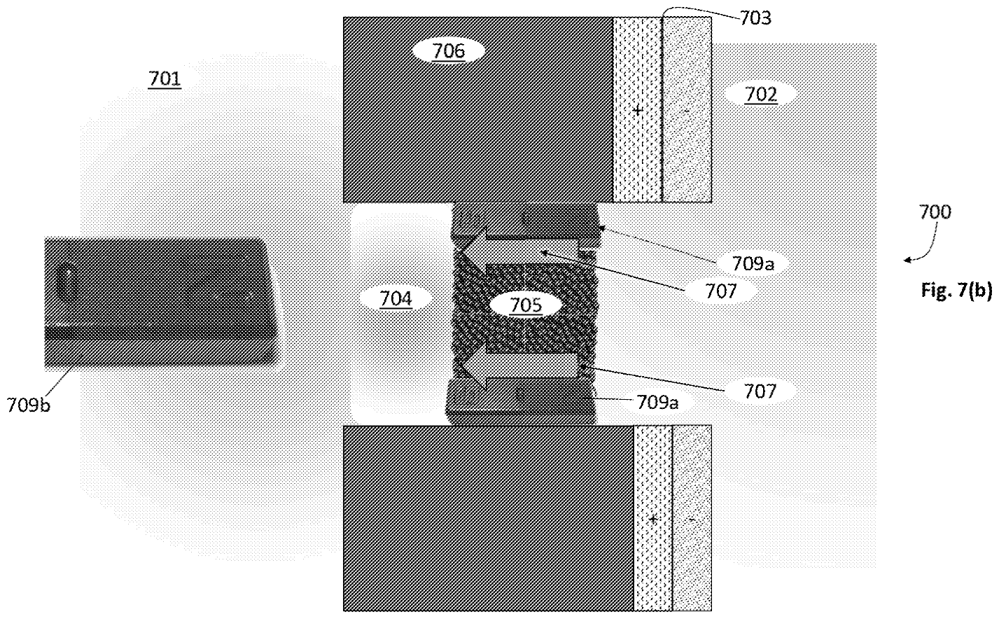

FIGS. 7A-B illustrate various exemplary methods for inducing motion of the plug relative to the barrier 606. FIG. 7A illustrates an apparatus 700 similar to that of FIGS. 6A-B. The apparatus includes a first solution compartment 701 and a second solution compartment 702 separated by a barrier 706. An electret material 703 is configured on barrier 706, on the side facing second solution compartment 702. Plug 705 is configured within an opening in barrier 706. Within first solution compartment 701, region 704 is a “focal zone” at which negatively charged material has accumulated, in order to be transferred to the other side of plug 705 to second solution compartment 702. These elements are similar to the equivalent elements as described in connection with FIGS. 6A-C, and, for brevity, elaborate descriptions of those elements are not repeated here.

Referring to FIG. 7A, a spring 708a is used to cause the relative motion of the plug 705, as shown by arrow 707. Extension of the spring 708a causes the spring 708 to push the plug 705. As the spring 708a pushes the plug, the solution starts to flow through the channels in the plug, thereby reducing the pressure of the solution in the first solution compartment and permitting continued advance of the spring 708a. The speed of the motion of the plug 705 depends on, inter alia, the spring constant, the resistance to the motion caused by the incompressible fluid, and, conversely, by the capability of the fluid to flow through the microchannels of the plug 705. To the extent that resistance to flow through the plug 705 is lower, and the force of the spring is higher, the plug 705 will advance faster. The timing of the motion of plug 705 may accordingly be set to allow the analyte species to accumulate in region 704 and transit to the other side of plug 705 as desired.

The spring 708a may be of any suitable geometry or type for achieving the effects described herein. In the illustrated embodiment, the spring 708a is a coil spring. In the alternative, the spring may be a cantilever spring, or an elastic material, such as rubber. The spring may be a shape memory alloy (SMA), that may be made to change its form (e.g., to expand) by applying a heating sequence, for example, or using any other suitable method.

FIG. 7B illustrates another embodiment of a method for actuating plug 705. In this embodiment, plug 705 is attached to a first magnet 709a, which may be of any relevant shape and dimensions. First magnet 709a may point its north pole in any preferred direction. In the illustrated example, the north pole faces generally toward the second solution compartment 702. A second magnet 709b, which may be inside or outside of the first solution compartment, will affect the first magnet 709a. If second magnet 709b has its north pole toward plug 705, the second magnet 709b causes attraction with the south pole of the first magnet 709a, and attracts plug 705, so that plug 705 starts moving in direction 707. In this case as well, the speed of movement depends on the flow resistance caused by the channels in the plug and the force exerted between the magnets. Obviously, the same result is achieved if the south pole of each magnet is pointing to the right in FIG. 70B instead of to the left. In addition, it is possible to place second magnet 709b in the second solution compartment 702, with its north pole pointing to the north pole of first magnet 709a. Thus, the poles are repelled one relative to the other, and the plug is pushed away from the magnet, instead of being attracted to it. In another alternative, one or both of the first magnet 709a and the second magnet 709b may be an electromagnet. Advantageously, this type of magnet may enable more controllable motion, albeit at the expense of the requirement of a source of current to energize the coils of the magnet.

Instead of a magnet, it is possible to use an electrostatic dipole and/or charges, such that magnet 709b may be exchanged for a dipole element such as an electret. For example, the north pole may be replaced by a positive electrostatic charge and the south pole by a negative electrostatic charge. The electrostatic charges are attracted or repelled relative to each other using electrostatic forces. The charge elements may each have a single charge, rather than a dipole, which is required for a magnet. Thus, instead of an electret, which is an electrostatic dipole, elements 709a and 709b may have opposite electrostatic charges, if an attractive force is preferred, or electrostatic charges having the same sign (both positive or negative), if repulsive forces are preferred. Any combination of the methods discussed here, including springs, magnetic, and electrostatic attractive or repulsive forces, may be utilized.

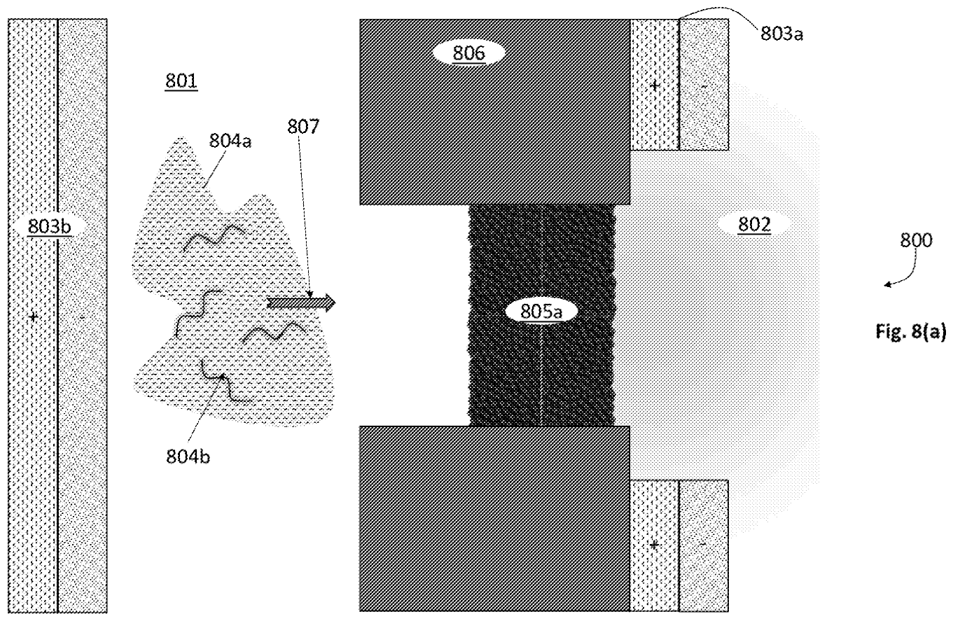

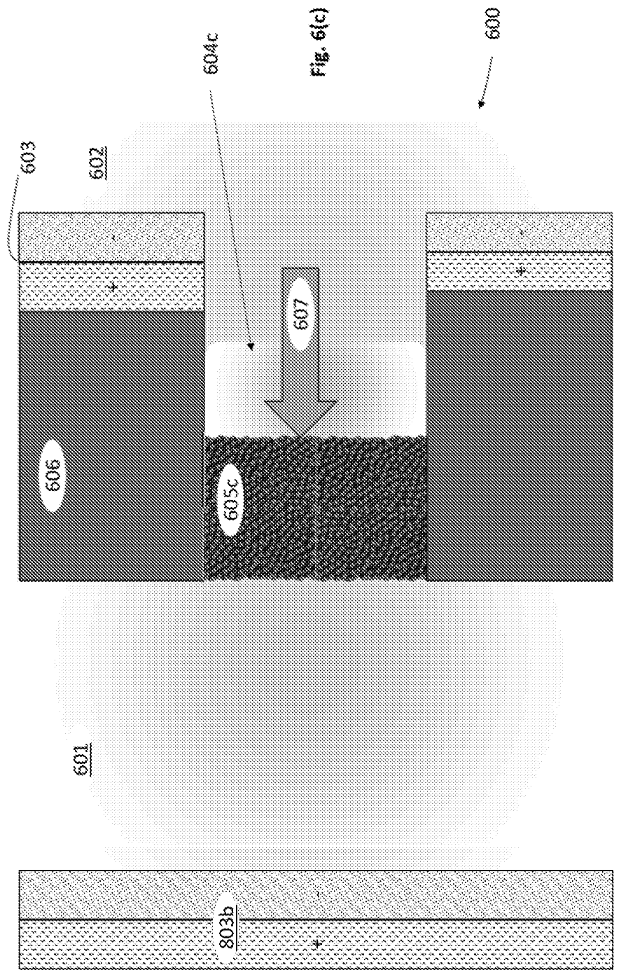

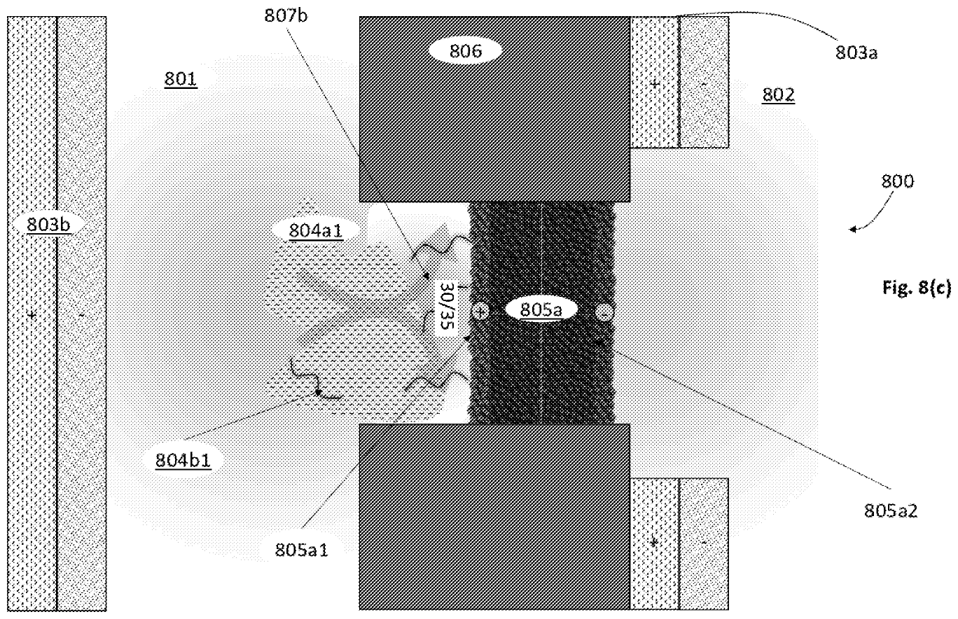

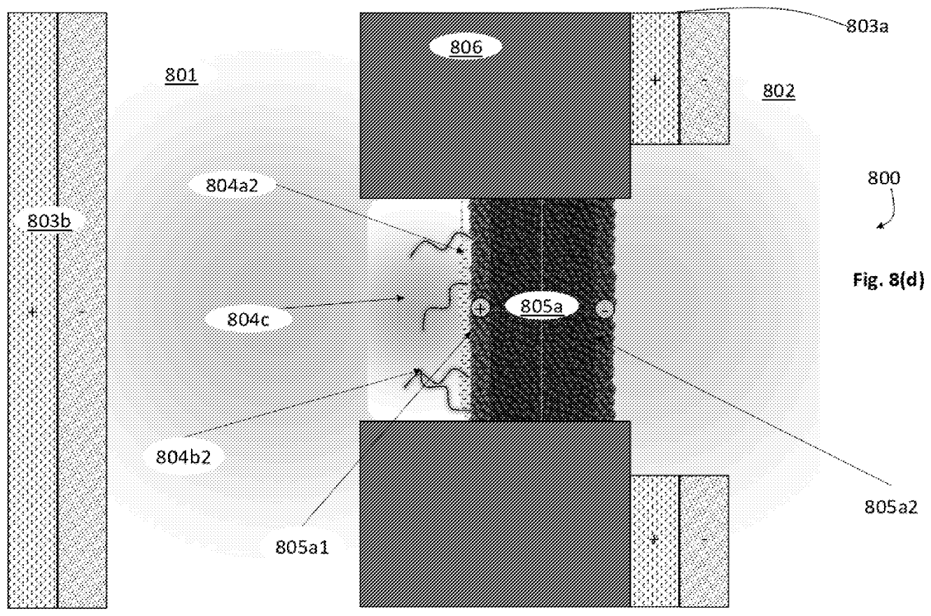

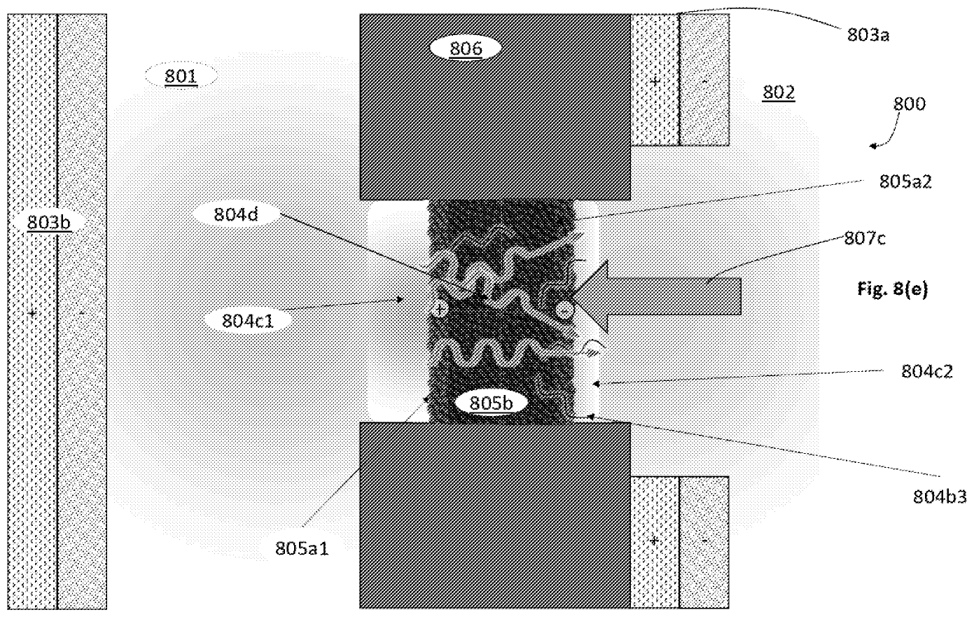

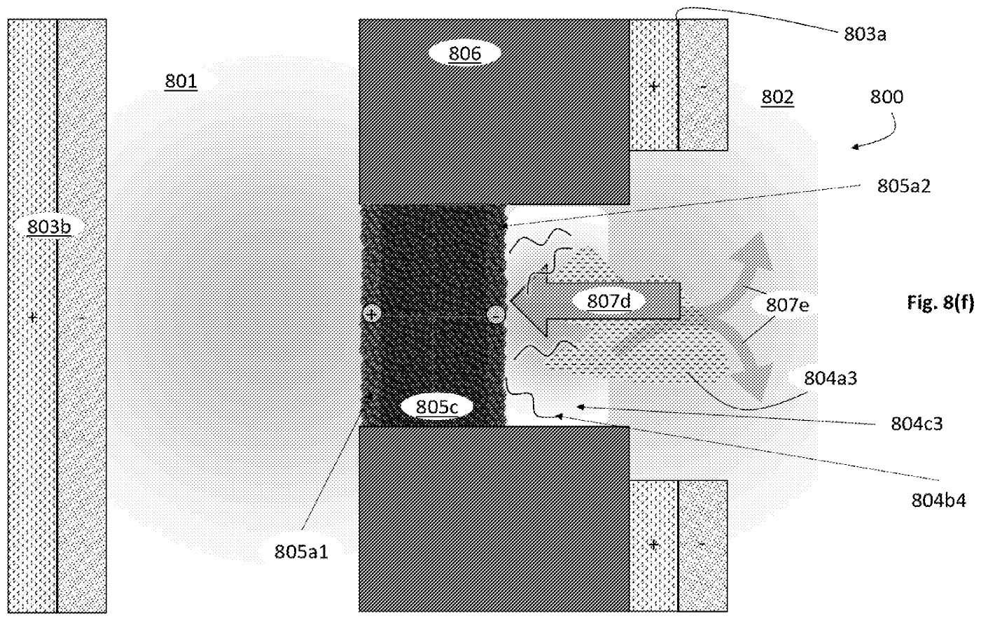

FIGS. 8A-F illustrate another embodiment of a plug, which is specifically made of conductive material, and a method of actuating movement of this conductive plug. Aside from being made of conductive material, the plug may be of similar construction to the embodiments of plugs described previously.

FIGS. 8A-F depict a close-up of a sensing device 800. The close-up is of a valve region. First solution compartment 801 and second solution compartment 802 are separated by barrier 806. An electret layer 803a may be placed on barrier 806. Electret 803a is arranged in such a manner so as not to interfere with the flow of fluid through the valve, and indeed plays no role in the transfer of fluid between the first and second solution compartments. Barrier 806 has an opening in which plug 805a is placed. The geometry of the openings on the separating structure 806 and the electret 803a might, but need not be, equal. Specifically, the opening in barrier 806 may be smaller than, equal to, or larger than the opening in the electret. In the illustrated embodiment, the electret layer 803a is placed on the side of the barrier 806 that points toward the second solution compartment 802, with the negative side toward the solution so as to repel negatively charged substances entering second solution compartment 802. In the illustrated embodiment, the opening in the electret layer 803a is larger than the opening of the separating structure 806. The electret layer 803a is drawn this way so as to emphasize that no electrostatic influence occurs in the region of the valve due to the electret 803a, and that electret 803a only exerts an influence on substances that are already within the second solution compartment. Those skilled in the art are well aware of other possible places and sizes of the electret.

In FIG. 8A, an electret layer 803b is shown at first solution compartment 801. This electret layer is illustrated schematically as being linear but may also be oriented in a curved manner, and as part of a larger electret configuration in order to focus the charged matter to a focal zone in the region of the valve, as previously discussed. In the illustrated example, the negative side of the electret layer 803b points to first solution compartment 801 and barrier 806. The first solution compartment 801 includes charged substances 804a, and among them specific molecules of interest 804b. The negatively charged molecules are repelled by the electrostatic effect of electret 803b and move in direction 807 to the opening in the separating structure 806, where plug 805a is present.

Referring to FIG. 8B, since plug 805a is conductive, the electret layer 803b, in addition to its influence on the charged particles in the first solution compartment 801, also induces a charge redistribution on plug 805a. The side of plug 805a that is closer to the negative side of electret 803b becomes charged with positive charges 805a1. As a consequence, the other side of the conductive plug 805a that faces the second solution compartment 802 will become charged with negative charge 805a2.

Referring now to FIG. 8C, the negatively charged species 804a1 and 804b1 proceed to move in direction 807b, and accumulate in a zone near the positive side of the plug. These species are both repelled by electret 803b, and also attracted by the induced charge 805a1 of the conductive plug 805a. The movement of the species occurs more quickly than movement of the plug, since species such as particles and molecules are small and mobile in a solution, relative to the plug. Thus, eventually, as illustrated in FIG. 8D, a substantial amount of negative substances 804a2 and within them molecules 804b2 will accumulate, forming a zone 804c, which contains the substances to be transferred to the second solution compartment.

As mentioned, due to electret 803b, a redistribution of charges occurs in plug 805a, together with the effects caused on substances 804, and a force is exerted on the plug charges 805a1 by the electret 803b. This force initially separated the positive charges of plug 805 to one side and the negative charges to the other side. This force further operates to attract plug 805, and induce a motion of the plug, in direction 807c, as shown in FIG. 8E. Although, in theory, the forces that induce the motion of the plug are present even before the accumulation of the molecules in zone 804c, in practice, the plug movement is apparent after the molecules have been accumulated. This is due to various physical factors, including, for example, frictional forces on the plug and the drag caused by fluid flowing through microchannels in the plug. As a consequence of the motion of the plug, the material 804c1 accumulated near the plug will transfer through the microchannels within the plug, and accumulate as material 804c2 on the other side of the plug. Negatively-charged molecules 804b3 will emerge on the side of the plug which has induced negative charges 805a2. Movement of the plug forces accumulated material 804c1 to transfer side to side, and by doing this induces the negative charges, which may have been slightly attracted by the induced positive charge at the portion of the plug facing the first solution compartment, to overcome the attraction based on the charge and move them to the other side of plug 805.

Eventually, as shown in FIG. 8F, the material will have passed from the first solution compartment side of the plug to the second solution compartment side of the plug, and have accumulated in region 804c3. Plug 805c completes its functioning as a valve while traveling in direction 807d. The charged substances at the other side of the plug are exposed to negative charges 805a2 caused by the induced dipole. These charges repel the transferred negative species (optionally, in conjunction with electret 803a). Accordingly, those negative species move in direction 807e toward the interior of second solution compartment 802.

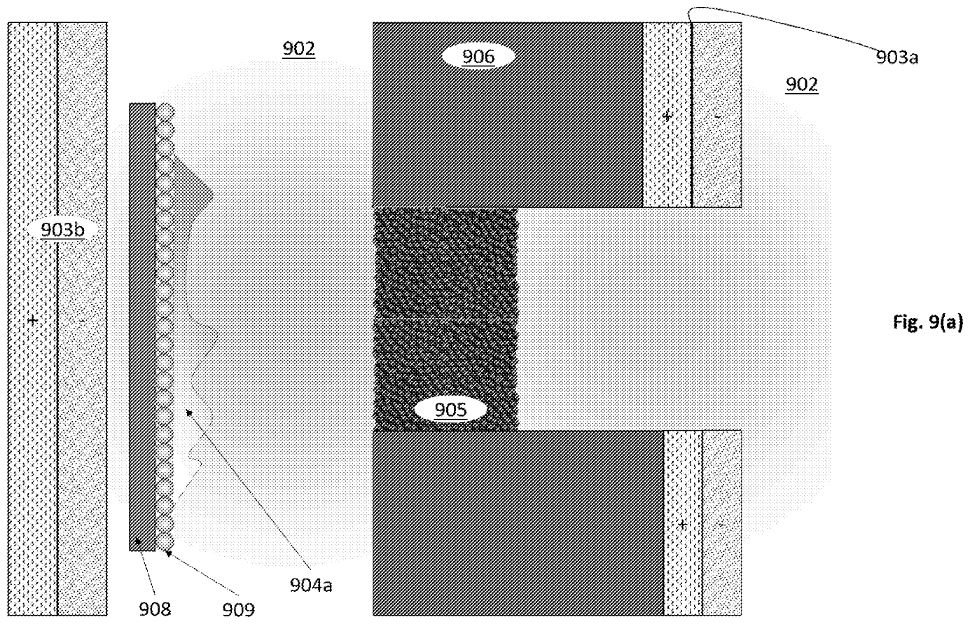

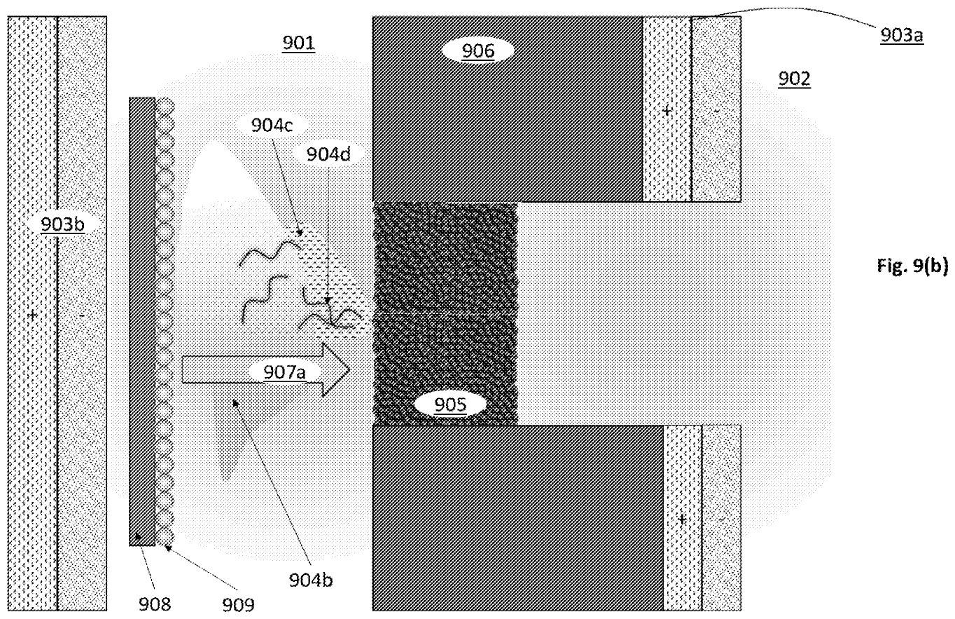

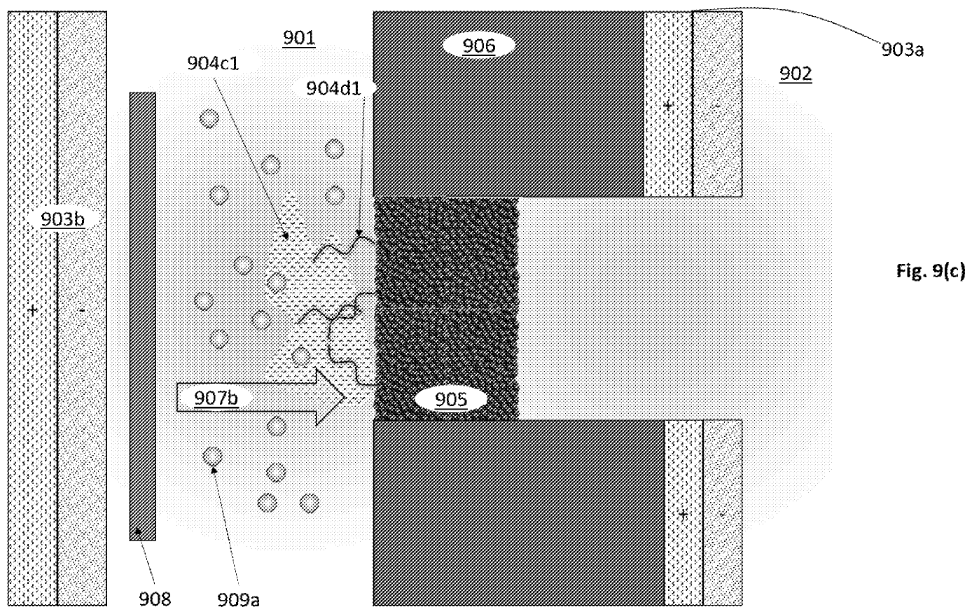

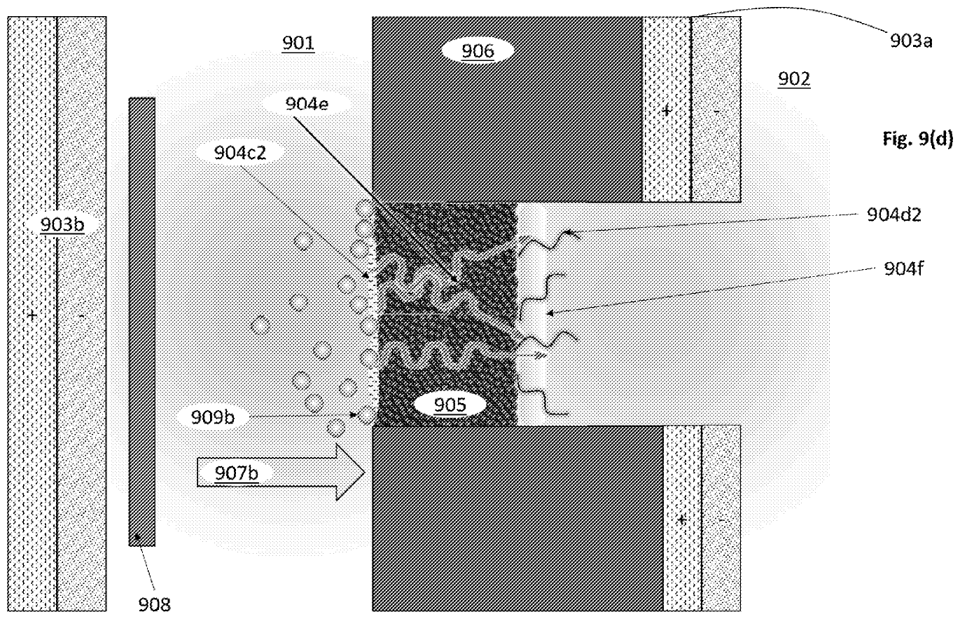

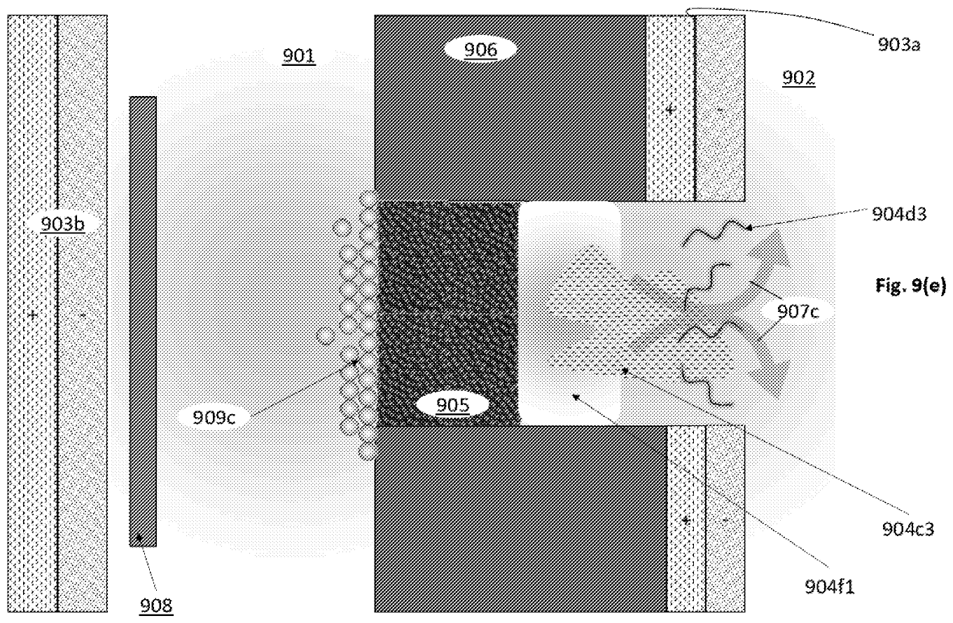

FIGS. 9A-E illustrate another embodiment of a valve with a plug. Device 360 has many features similar to the previous embodiments. For purposes of brevity, they are recited here only briefly. A first solution compartment 901 and a second solution compartment 902 are separated by a barrier 906. For purposes of this illustration, barrier 906 is described as being of a nonconductive or isolating material. Electret material 903a is on the side of barrier 906 facing the second solution compartment 902. Electret layer 903b is configured within the first solution compartment 901 and may be shaped to direct the charged material to a focal zone, as discussed. Electret layer 903b has its negatively charged side towards the first solution compartment and valve, so as to induce travel of negatively charged material to the valve.

Plug 905 is configured within a hole in barrier 90, and serves as a valve connecting the first and second solution compartments. In the illustrated embodiment, unlike the previous embodiments involving a plug, plug 905 is stationary relative to barrier 906, and in principle, may be fixed to barrier 906.

Referring to FIG. 9A, a sample is introduced into the solution. As previously discussed, the sample may be introduced in various ways, including directly into an opening on the vial, or through placement in the cap that seals the opening of the vial. In the illustrated embodiment, the sample is placed in a sample holder which is inserted into the solution. The sample holder is comprised of a substrate 908 and a layer of beads 909. Beads 909 may be of any suitable shape, such as spherical, pyramidal, or block-like. The beads 909 may be solid or hollow inside, and may be made of multiple layers. The beads are immobilized by use of a dissolvable adhesive. Sample 904a is placed on the beads 909. If the beads are bonded so as to form a substantially rigid surface resembling a substrate able to hold the sample, the separate substrate layer 908 is not necessary.

Beads 909 are made, at least in part, of a material having electric charge. In the illustrated embodiment, the charge of the beads is negative, as is the charge of the species that is to be moved in the solution.

Referring to FIG. 9B, once the sample holder with the sample is introduced into the solution, chemical reactions start to take place, as explained above in connection with FIGS. 3A-P. These reactions dissolve the sample and separate it into substances 904b, and specifically negatively charged substance 904c, and within them, potentially, specific molecules or species of interest 904d. The electret layer induces movement of the charged molecules to the plug. Due to the effect of the negatively charged side of electret 39B, and, potentially, due to the negatively charged beads 909, all the negatively charged species will start to move in direction 907a towards plug 905.

Simultaneously, the adhesive that bonds beads 909 to each other and to the optional substrate 908 starts to dissolve or lose hold, due to the effect of the solution on the agent bonding the beads. The beads are released to solution 909a. The electret layer 903b causes movement of the released beads 909 to the plug. All of the negatively charged elements, including beads 909a, the negative species released to the solution from sample 904c1, including some samples of interest 904d1, move in direction 907b toward plug 905. The negative species 904c1, 904d1 are released earlier and are typically smaller and more agile than the beads 909a; thus, they will reach plug 905 earlier than the larger and relatively slower beads 909a.

Referring to FIG. 9D, when the species arrive at the plug, they accumulate at side 904c2. Due to the electrostatic repulsion forces caused by the electret layer and the accumulated charged beads 909B, the charged molecules will diffuse through channels 904e in plug 905 to the other side of the plug. Unlike in the embodiments of FIGS. 6A-6C, 7A-7B, and 8A-8F, the fluid remains on the first solution compartment side of the plug 905, and only the species transit the plug 905. The electrostatic forces cause movement only for the species. The species accumulate in zone 904f, including species of interest 904d2, if present. At the same time, the rest of the charged species continue their motion 907b towards the plug including beads 909b, which begin to accumulate near plug 905.

Referring to FIG. 9E, eventually, most of the negatively charged species are transferred through the plug and accumulate in region 904f1 at the second solution compartment 902 side of the plug. The beads 909c, being larger than the passages of the plug 905, cannot transit through the plug 905, and therefore accumulate on it at the first solution compartment 901 side of the plug 905, effectively blocking further passage of negative charges in general (due to the repulsion effect) as well as other species. The beads become arranged along the surface of the plug according to the combined electrostatic effects of the electret layer and the induced dipole of the plug. The design of the charge in the electret, size of the beads, and distribution of channels in the plug, dictate an optimal flow of species on one hand, and the effective blocking of non-intended species through the plug on the other hand. In addition to the electret 903b, the negatively charged field of the beads forms a wall 909c, which further repels the negative species being accumulated in region 904f1, and thus move the negatively charged species 904c3, and the specific species 904d3 in particular, in direction 907c, toward second solution compartment 902.

Optionally, beads 909 are hollow. The hollow beads store a material. The surface of the beads may be made, at least in part, of a dissolvable material. When the beads are released into the solution, the walls start to disintegrate such that, when the released beads have accumulated on the surface of plug 905 so as to form wall 909c blocking the passages within plug 905, openings occur in the surface of the beads, thus releasing the material from the interior of the beads. This released material flows out of the beads and creates a membrane or other structure that bonds the beads together. The bonded beads block passage of charged particles backward through the plug. The dissolvable material of the surface of the beads may be selected, and for example may be applied at a suitable thickness, so that this dissolution fully opens the openings only after the beads have accumulated on the surface of plug 905. In addition, it is well understood by those skilled in the art that the plug structure and the microchannel and passages in it can be made such that they filter the species passing through them according to size such that only molecules or particles of a predefined size can pass through from one compartment to the other thus increasing the selection capabilities of this type of plug.

What is claimed is:

1. An apparatus for transferring charged molecules between a first solution compartment and a second solution compartment, comprising:
a barrier arranged between the first and second solution compartments;
a valve configured within the barrier; and
a plurality of electrets within at least one of the first and second solution compartments, the plurality of electrets sized and shaped to accumulate the charged molecules at a focal zone on a first solution compartment side of the valve.

2. The apparatus of claim 1, further comprising a cavity within the valve for receipt of the charged molecules, and wherein rotation of the cavity relative to the barrier causes delivery of the charged molecules within the cavity from the first solution compartment side of the valve to a second solution compartment side of the valve.

3. The apparatus of claim 2, wherein the valve is comprised of a conductive material and includes a conductive extension opposite the cavity, and wherein first and second electrets are configured on respective portions of the barrier facing the second solution compartment, wherein, when the cavity faces the first solution compartment, the conductive extension is configured parallel to the first electret, thereby inducing a charge gradient within the valve that attracts the charged molecules to the cavity; and wherein, following rotation of the valve such that the cavity faces the second solution compartment, the conductive extension is parallel to the second electret, thereby inducing a charge gradient within the valve that repels the charged molecules from the cavity.

4. The apparatus of claim 3, further comprising a first barrier section made of isolating material configured to separate between the first electret and the valve extension, and a second barrier section made of isolating material configured to separate between the second electret and the valve extension.

5. The apparatus of claim 1, wherein the valve comprises a plug that is movable relative to the barrier, wherein the plug comprises microchannels that permit passage of fluid therethrough, wherein movement of the plug relative to the barrier toward an interior of the first solution compartment reduces a volume of the first solution compartment, thereby inducing flow of the charged molecules through the microchannels to the second solution compartment.

6. The apparatus of claim 5, wherein the plug is comprised of an agglomerate of one or more of: microbeads, microspheres, ceramic materials, or powdered materials.

7. The apparatus of claim 5, further comprising a spring configured on the second solution compartment side of the plug, wherein extension of the spring causes the movement of the plug toward the interior of the first solution compartment.

8. The apparatus of claim 5, further comprising a first magnet attached to the plug, and a second magnet fixed at an opposite end of the first solution compartment relative to the plug, wherein a magnetic attraction between the first magnet and the second magnet causes movement of the plug.

9. The apparatus of claim 5, further comprising a first electrostatic charge attached to the plug, and a second electrostatic charge fixed at an opposite end of the first solution compartment relative to the plug, wherein an electrostatic force between the first electrostatic charge and the second electrostatic charge causes movement of the plug.

10. The apparatus of claim 5, wherein the plug is made of conductive material.

11. The apparatus of claim 10, wherein the plurality of electrets comprise an electret layer configured within the first solution compartment, wherein the electret layer is configured to: (1) repel the charged molecules from the end of the first solution compartment toward the plug, (2) induce a dipole within the plug; and (3) following inducing of the dipole within the plug, exert an electrostatic force on the plug to thereby: draw the plug toward an interior of the first solution compartment, reduce the volume of the first solution compartment, and induce flow of the charged molecules through the microchannels to the second solution compartment.

12. The apparatus of claim 11, wherein the induced dipole of the plug repels the charged molecules within the second solution compartment from the plug.

13. The apparatus of claim 1, wherein:
the valve comprises a plug that comprises microchannels that permit passage of fluid therethrough;
the plug is made of conductive material;
the plurality of electrets comprise an electret layer configured within the first solution compartment, wherein the electret layer is configured to: (1) repel the charged molecules from the end of the first solution compartment toward the plug, and (2) induce a dipole within the plug;
the apparatus further comprises a sample holder comprised of a plurality of beads having an electric charge, the plurality of beads being bonded together with a dissolvable adhesive;
wherein, upon introduction of the sample holder holding the sample into the first solution compartment:
a solution within the first solution compartment dissolves the adhesive, to thereby release the plurality of beads into the first solution compartment; and
the electret induces movement of the charged molecules to the plug, diffusion of the charged molecules through the plug, and movement of the released beads to the plug.

14. The apparatus of claim 13, wherein the beads comprise a surface that is at least partially dissolvable and an adhesive configured within the surface, wherein dissolution of the surface releases the inner adhesive, allowing the beads to thereby bond together at the plug to block passage of the charged molecules backward through the plug.

15. The apparatus of claim 1, wherein the first solution compartment contains one or more chemicals configured to break down a biological sample into double-stranded or single-stranded DNA.

16. The apparatus of claim 1, further comprising, within the second solution compartment, a sensor configured to test for the presence of specific single-stranded strands of DNA.

17. A method of transferring charged molecules between a first solution compartment and a second solution compartment, comprising:
delivering the charged molecules to a focal zone of a valve configured within a barrier between the first and second solution compartments, through operation of a plurality of electrets within at least one of the first and second solution compartments; and
transferring the charged molecules through the valve.

18. The method of claim 17, wherein the valve includes a cavity therein for receipt of the charged molecules, and further comprising rotating the cavity relative to the barrier to thereby deliver the charged molecules within the cavity from a first solution compartment side of the valve to a second solution compartment side of the valve.

19. The method of claim 18, wherein the valve member is comprised of a conductive material and includes a conductive extension opposite the cavity, and wherein first and second electrets are configured on respective portions of the barrier facing the second solution compartment, wherein when the cavity faces the first solution compartment, the conductive extension is configured parallel to the first electret, thereby inducing a charge gradient within the valve that attracts the charged molecules to the cavity; and wherein, following the rotating step, the valve extension is parallel to the second electret, thereby inducing a charge gradient within the valve that repels the charged molecules from the cavity.

20. The method of claim 19, wherein the valve comprises a plug that is movable relative to the barrier, wherein the plug comprises microchannels that permit passage of fluid therethrough, and wherein the transferring step comprises moving the plug relative to the barrier toward an interior of the first solution compartment, to thereby reduce a volume of the first solution compartment, inducing flow of the charged molecules through the microchannels to the second solution compartment.

21. The method of claim 20, further comprising causing movement of the plug through extension of a spring.

22. The method of claim 20, further comprising causing movement of the plug through a magnetic attraction between a first magnet attached to the plug and a second magnet fixed at an opposite end of the first solution compartment relative to the plug.

23. The method of claim 20, further comprising causing movement of the plug through an electrostatic force induced between a first electrostatic charge attached to the plug, and a second electrostatic charge fixed at an opposite end of the first solution compartment relative to the plug.

24. The method of claim 20, wherein the plug is made of conductive material.

25. The method of claim 24, wherein the plurality of electrets comprise an electret layer configured within the first solution compartment, and the method further comprises, through action of the electret layer: repelling the charged molecules from the end of the first solution compartment toward the plug, inducing a dipole within the plug; and following inducing of the dipole within the plug, exerting an electrostatic force on the plug to thereby draw the plug toward the electret layer, reduce the volume of the first solution compartment, and induce flow of the charged molecules through the microchannels to the second solution compartment.

26. The method of claim 17, wherein the valve comprises a plug that comprises microchannels that permit passage of fluid therethrough; the plug is made of conductive material; and the plurality of electrets comprise an electret layer configured within the first solution compartment, wherein the electret layer is configured to: (1) repel the charged molecules from the end of the first solution compartment toward the plug, and (2) induce a dipole within the plug; and the method further comprises:
receiving a sample within the first solution compartment on a sample holder comprised of a plurality of beads having an electric charge, the plurality of beads being bonded together with a dissolvable adhesive,
dissolving the adhesive with a solution within the first solution compartment, to thereby release the plurality of beads into the first solution compartment; and
with the electret layer, inducing movement of the charged molecules to the plug, diffusion of the charged molecules through the plug, and movement of the released beads to the plug.

27. The method of claim 17, further comprising, within the first solution compartment, breaking down biological material into double-stranded or single-stranded DNA.

28. The method of claim 17, further comprising, within the second solution compartment, testing a sample for the presence of specific single-stranded DNA strands.
